(19) United States
(12) Patent Application Publication
(10) Pub. No.: US 2004/0121360 A1
(43) Pub. Date: Jun. 24, 2004

(54) Methods, platforms and kits useful for identifying, isolating and utilizing nucleotide sequences which regulate gene exoression in an organism

(76) Inventors: Hagai Karchi, Moshav Sitriya (IL); Rafael Meissner, Rehovot (IL); Gil Ronen, Shimshon (IL)

Correspondence Address: Anthony Castorina, G E Ehrlich, Suite 207, 2001 Jefferson Davis Highway, Arlington, VA, 22202, US

(21) Appl. No.: 10/471,606

(22) PCT Filed: Mar. 31, 2002
(86) PCT No.: PCT/IL02/00267

(51) Int. Cl. C12Q 1/68

(52) U.S. Cl. 435/6

(57) ABSTRACT

A method of generating genotypic and possibly phenotypic variation in an organism is provided. The method is effected by (a) isolating at least one non-coding nucleic acid sequence from a genome of the organism; and (b) genetically transforming the organism with the at least one non-coding nucleic acid sequence to thereby generate genotypic and possibly phenotypic variation in the organism.

### FIELD AND BACKGROUND OF THE INVENTION

The present invention relates to methods, platforms and kits for identifying and isolating non-coding genomic sequences which regulate gene expression in an organism. Embodiments of the present invention relate to methods of isolating and utilizing non-transcribed genomic sequences for generating genotypic and possibly phenotypic variation in the organism and for identifying and characterizing regulatory sequences participating in biological pathways.

The availability of high-throughput sequencing tools has led to the generation of a number of large databases, each including sequence data of a specific organism. For example, the genomes of several model organisms including that of the model plant species Arabidopsis have now been completely sequenced (The Arabidopsis Genome Initiative, Nature 2000, 408:796).

Considerable volumes of information are also available regarding transcribed nucleic acid sequences. As of the time of writing, the NCBI database of ESTs (http://www.ncbi.nlm.nih.gov/dbEST/) contained 7,729,552 entries. Furthermore, an enormous amount of data enters the database every month. For example, in the three months preceding the time of writing, 851,352 new entries were added to the database, representing an increase of 11%, about two-thirds of which representing human sequences and the remaining representing a variety of other organisms. Classification of this database by organism shows a huge variation in the number of ESTs that are produced for each organism; ranging from 3,397,913 ESTs for human to 1 EST for Trichoplusia ni (cabbage looper).

The availability of complete genomic sequences of organisms constitutes a powerful tool for research aimed at elucidating the genetic mechanisms underlying the development and growth of such organisms.

For example, such sequence information can be used to enhance the capacity to generate plant varieties having desired characteristics. This is clearly of tremendous potential impact in the fields of agriculture, pharmacology, textiles, horticulture and all other industries involving the use of plants and plant derived products.

Current research approaches, including approaches seeking to elucidate genetic mechanisms responsible for generation of phenotypic diversity, currently focus on the transcribed fraction of the genome which, in the case of Arabidopsis, constitutes only 10-20% of the entire genome.

Non-coding sequences, however, encode DNA regulatory elements (DREs) which play a critical role in determining the phenotype of organisms, including plants, since such sequences function as the master switches of gene expression. Such DREs comprise regulatory elements such as promoters, enhancers, suppressors, silencers and locus control regions.

Promoters are generally located adjacent to transcriptional start sites and function in an orientation-dependent manner while enhancer and suppressor elements, which modulate the activity of promoters, are flexible with respect to their orientation and distance from transcriptional start sites and, as such, can also be located within introns.

Gene regulatory sequences are specifically bound by gene specific transcription factors (TFs) and, in many cases, a complex of other gene specific accessory proteins, the sum of which determine the rate, cell type specificity and developmental stage specificity of gene transcription as well as transcriptional responses to given physiological conditions.

Thus, identifying gene regulatory sequences and, in particular, elucidating their effects on gene expression, is central to understanding and manipulating the complex and powerful gene regulatory networks of organisms.

The potential complexity of gene regulatory networks is highlighted, for example, in the sea urchin Endo 16 gene, encoding a polyfunctional embryonic secretory protein, whose upstream flanking region contains at least 33 TF binding sites grouped into five different modules (Yuh C H et al., Science 1998, 279:1896). Such complexity underscores the importance and flexibility of gene regulatory networks and provides a rich source of potential control points for gene regulation.

It has been suggested that the same set of structural genes are apt to be involved in the formation of homologous biological structures in evolutionarily related organisms, such as, for example, the hooves of ungulates and the fingers of primates, anatomic elements which are functionally and anatomically distinct from one another (Ohno S, J Human Evol. 1971, 1:651). As such, variations in gene regulation can be responsible for generation of varying structures from similar structural genes.

It has been shown that many of the same gene families exist in most animals and regulate major aspects of body patterning with morphological variations arising as a result of alterations in gene regulation. For example, all four arthropod classes share nearly identical sets of Hox genes despite their great morphological diversity and distant (>5.4×10⁸ years) evolutionary divergence from a common ancestor (Carroll S B, Cell 2000, 101:577). These genes play a key role in directing the identity and formation of the primary axial segments of the body plan of animals during development. Comparative analysis of Hox gene expression in mice and chicks has shown that primary differences in expression patterns of these genes appear in the form of shifts in expression along the primary body axis as a result of differences in their regulatory elements (Carroll et al., In: “Molecular Genetics And The Evolution Of Animal Design”, Malden, M A., Blackwell Scientific (2000); Belting H G et al., Proc Natl Acad Sci USA. 1998, 95:2355; Cohn M J and Tickle C, Nature 1999, 399:474; Weatherbee S D and Carroll S B, Cell 1999, 97:283).

An example of variation within the same species resulting from varying regulation of similar structural genes is also found in the bristle pattern of the fruit fly Drosophila melanogaster, a feature which varies between individuals and populations. Several loci involved in controlling bristle number have been mapped to non-transcribed regions (Mackay T F C. Bioassays 1996, 18:113; Long A D. et al., Genetics 1998, 149:999). Thus, modifications in regulatory elements can regulate the levels, patterns and timing of gene expression in animals.

The phenotype of plants is also determined by the nature of regulatory elements controlling gene expression. For example, it has been shown that the genomes of different species of cereal plants encode similar genes while varying in numbers of non-transcribed, repetitive DNA sequences (Messing and Llaca, Proc Natl Acad Sci USA. 1998, 5:2017).

Furthermore, the fact that plants tolerate insertions and other sequence rearrangements within non-coding regions implies that these regions are candidates for mutational hot spots (Wessler S et al., Curr Opin Genet Dev. 1995, 5:459). This has been confirmed by studies which compared two plants with dramatically differing phenotypes; maize and the wild Mexican grass teosinte from which it was domesticated 5,000-10,000 years ago (Doebley J et al., Nature 1997, 386:485; Wang R L et al., Nature 1999, 398:236). These studies showed that various maize and teosinte sequences encoding the teosinte branched 1 (tb1) gene, a locus responsible for some of the phenotypic differences between these two species, encode gene products of identical amino acids. It was further shown that in the process of domesticating maize from teosinte, selection was primarily dependent upon the regulatory elements of this gene (Wang R L et al., Nature 1999, 398:236). These observations therefore demonstrate that modifications in gene regulatory sequences can be used to generate novel plant species having highly desirable characteristics, as has been the case with maize.

Several studies have similarly demonstrated that rearrangements in non-coding sequences can produce novel patterns of gene expression and thereby create novel phenotypes. For example, it was demonstrated that a spontaneously arising fusion protein of a metabolic enzyme and the homeodomain protein Let6 in tomato led to overexpression of the fusion protein relative to wild-type Let6, and that this resulted in the conversion of a unipinnate leaf phenotype to well-integrated tri- or tetra-pinnate compound leaf phenotypes (Chen J J, Plant Cell 1997, 9:1289).

Thus, elucidation of the functions of regulatory sequences of plants, as well as of other organisms, is of great importance for both research and commercial applications.

However, very little information is currently available regarding the functions of regulatory sequences due to the lack of research tools capable of systematically investigating, with genomic scope, the functions and potential uses of such regulatory sequences.

There is thus a widely recognized need for, and it would be highly advantageous to have, a method of systematically identifying, cloning and elucidating the function of regulatory sequences, such as those found in non-coding regions of the genome, and of utilizing such sequences to generate genotypic and, optionally phenotypic, variation in organisms, such as, for example, plants.

### SUMMARY OF THE INVENTION

According to one aspect of the present invention there is provided a method of generating genotypic and possibly phenotypic variation in an organism comprising: (a) isolating at least one non-coding nucleic acid sequence from a genome of the organism; and (b) genetically transforming the organism with the at least one non-coding nucleic acid sequence to thereby generate genotypic and possibly phenotypic variation in the organism.

According to another aspect of the present invention there is provided a method of identifying novel gene expression regulatory sequences comprising: (a) isolating at least one non-coding nucleic acid sequence from a genome of an organism; (b) transforming the organism with an expression cassette including the at least one non-coding nucleic acid sequence covalently linked to a reporter nucleic acid sequence; and (c) monitoring reporter activity, the reporter activity being indicative of a presence of a regulatory sequence in the at least one non-coding nucleic acid sequence.

According to still another aspect of the present invention there is provided a method of generating a database of putative regulatory sequences of a genome of an organism comprising: (a) computationally clustering transcribed nucleic acid sequences of the organism to thereby obtain a plurality of clusters; (b) computationally generating contigs from at least a subset of the plurality of clusters; (c) computationally aligning the contigs with the genomic nucleic acid sequences of the organism to thereby obtain inter-contig region sequences of the genome of the organism; and (d) storing the inter-contig region sequences of the genome of the organism in a database.

According to an additional aspect of the present invention there is provided a computer readable media comprising as retrievable records data pertaining to a plurality of nucleic acid sequences, each of the plurality of nucleic acid sequences representing an inter-contig region sequence of a genome of a single organism.

According to yet an additional aspect of the present invention there is provided a nucleic acid construct library comprising a plurality of nucleic acid constructs each including a specific non-coding nucleic acid sequence of an organism and devoid of coding sequences of the organism.

According to still an additional aspect of the present invention there is provided a method of determining the minimal number of expressed sequence tags (ESTs) needed for constructing substantially all of the coding sequences of a genome of an organism, the method comprising: (a) predicting the number of genes present in the genome of the organism, the number of genes being represented by N; (b) obtaining a product of N(ln(N)+C), wherein C=0.5772, the product being the minimal number of ESTs needed for constructing substantially all of the coding sequences of a genome of an organism.

According to a further aspect of the present invention there is provided a kit comprising a plurality of primer pairs, each of the primer pairs being complementary with nucleic acid sequences flanking a specific inter-contig region sequence of a genome of an organism, such that the kit being useful for amplifying a plurality of inter-contig region sequences of the genome of the organism.

According to yet a further aspect of the present invention there is provided a method of identifying putative regulatory sequences comprising: (a) computationally identifying inter-contig region sequences of at least two distinct organisms; and (b) computationally comparing the inter-contig region sequences of the at least two distinct organisms to thereby identify non-redundant sequences, the non-redundant sequences being putative regulatory sequences.

According to still a further aspect of the present invention there is provided a method of generating genotypic and possibly phenotypic variation in an organism comprising: (a) isolating at least one non-coding nucleic acid sequence from a genome of the organism; (b) covalently linking the at least one non-coding nucleic acid sequence to a known coding sequence to thereby generate an expression cassette; and (b) genetically transforming the organism with the expression cassette to thereby generate genotypic and possibly phenotypic variation in the organism.

According to another aspect of the present invention there is provided a method of uncovering regulatory sequences functional in a biological pathway of an organism, the method comprising: (a) isolating non-coding nucleic acid sequences from a genome of the organism; (b) covalently linking each of the non-coding nucleic acid sequences to a reporter coding sequence to thereby generate a plurality of expression cassettes; (c) genetically transforming a plurality of organisms with the plurality of the expression cassettes; (d) inducing activation of the biological pathway in the plurality of organisms; and (e) monitoring reporter activity in the plurality of organisms prior to, and following, step (d), to thereby determine the presence or absents of a regulatory sequence functional in the biological pathway in each of the non-coding nucleic acid sequences.

According to yet another aspect of the present invention there is provided a method of generating phenotypic variation in an organism comprising: (a) isolating non-coding nucleic acid sequences from a genome of the organism; (b) generating a plurality of organisms genetically transformed with the non-coding nucleic acid sequences; and (c) isolating an organism of the plurality of organisms which exhibits phenotypic variation.

According to still another aspect of the present invention there is provided a method of generating phenotypic variation in an organism comprising: (a) isolating non-coding nucleic acid sequences from a genome of the organism; (b) combinatorially shuffling regions derived from the non-coding nucleic acid sequences, to thereby generate combinatorial non-coding nucleic acid sequences; (b) generating a plurality of organisms genetically transformed with the combinatorial non-coding nucleic acid sequences; and (c) isolating an organism of the plurality of organisms which exhibits phenotypic variation.

According to a further aspect of the present invention there is provided a computing platform for identifying inter-contig region sequences of an organism and for generating primer sequences for amplifying the inter-contig region sequences, the computing platform comprising a processing unit being for: (a) computationally comparing data pertaining to transcribed nucleic acid sequences of an organism with data pertaining to genomic sequences of the organism to thereby generate data pertaining to inter-contig sequences of the organism; and (b) automatically generating primer sequences suitable for amplifying the inter-contig sequences of the organism.

According to further features in preferred embodiments of the invention described below, the at least one non-coding nucleic acid sequence is isolated from an inter-contig region of the genome.

According to still further features in the described preferred embodiments, the organism is a plant.

According to still further features in the described preferred embodiments, isolating the at least one non-coding nucleic acid sequence is effected by: (i) computationally clustering transcribed nucleic acid sequences of the organism to thereby obtain a plurality of clusters; (ii) computationally generating contigs from at least a subset of the plurality of clusters; (iii) computationally aligning the contigs with the genomic nucleic acid sequences of the organism to thereby identify inter-contig region sequences of the genome of the organism; and (iv) amplifying at least one of the inter-contig region sequences to thereby obtain the at least one isolated non-coding nucleic acid sequence.

According to still further features in the described preferred embodiments, the transcribed sequences are selected from the group consisting of EST sequences, cDNA sequences, mRNA sequences and preanalyzed genomic sequences.

According to still further features in the described preferred embodiments, the method of generating genotypic and possibly phenotypic variation in an organism further comprising assigning to the contigs a score according to at least one parameter selected from the group consisting of: (a) the number of the transcribed nucleic acid sequences clustered; (b) the percent homology of nucleotide sequences of the contigs to nucleotide sequences of known transcription factors; (c) the percent homology of nucleotide sequences of the contigs to nucleotide sequences of selected genes of interest; (d) the number of expression libraries from which the contigs were generated; (e) the number of types of expression libraries from which the contigs were generated; (f) the number of RNAs comprised in the plurality of clusters; (g) the length of the contig; (h) a user-defined quality score; (i) the type of tissues from which the transcribed nucleic acid sequences were derived; (j) the developmental stage of the tissues from which said transcribed nucleic acid sequences were derived (k) the growth conditions of the tissue from which said transcribed nucleic acid sequences were derived; and (l) the number of clusters of said transcribed nucleic acid sequences generated by the library from which said contigs are derived.

According to still further features in the described preferred embodiments, the expression cassette further includes a promoter sequence upstream of the reporter nucleic acid sequence.

According to still further features in the described preferred embodiments, the transcribed nucleic acid sequences are selected from the group consisting of EST sequences, mRNA sequences and preanalyzed genomic sequences.

According to still further features in the described preferred embodiments, the method of identifying novel gene expression regulatory sequences, further comprising assigning to the contigs a score according to at least one parameter selected from the group consisting of: (a) the number of the transcribed nucleic acid sequences clustered; (b) the percent homology of nucleotide sequences of the contigs to nucleotide sequences of known transcription factors; (c) the percent homology of nucleotide sequences of the contigs to nucleotide sequences of selected genes; (d) the number of expression libraries from which the contigs were generated; (e) the number of types of expression libraries from which the contigs were generated; (f) the number of RNAs comprised in the plurality of clusters; (g) the length of the contig; (h) the types of methods whereby the transcribed nucleic acid sequences were derived; (i) the type of tissues from which the transcribed nucleic acid sequences were derived; (j) the developmental stage of the tissues from which said transcribed nucleic acid sequences were derived (k) the growth conditions of the tissue from which said transcribed nucleic acid sequences were derived; and (l) the number of clusters of said transcribed nucleic acid sequences generated by the library from which said contigs are derived.

According to still further features in the described preferred embodiments, the method of generating a database of putative regulatory sequences of a genome of an organism, further comprising: (e) computationally clustering the inter-contig region sequences of the genome of the organism to thereby identify and group non-redundant sequences.

According to still further features in the described preferred embodiments, the method of generating a database of putative regulatory sequences of a genome of an organism, further comprising assigning to the contigs a score according to at least one parameter selected from the group consisting of: (a) the number of the transcribed nucleic acid sequences clustered; (b) the percent homology of nucleotide sequences of the contigs to nucleotide sequences of known transcription factors; (c) the percent homology of nucleotide sequences of the contigs to nucleotide sequences of selected genes; (d) the number of expression libraries from which the contigs were generated; (e) the number of types of expression libraries from which the contigs were generated; (f) the number of RNAs comprised in the plurality of clusters; (g) the length of the contig; (h) the types of methods whereby the transcribed nucleic acid sequences were derived; (i) the type of tissues from which the transcribed nucleic acid sequences were derived; (j) the developmental stage of the tissues from which said transcribed nucleic acid sequences were derived (k) the growth conditions of the tissue from which said transcribed nucleic acid sequences were derived; and (l) the number of clusters of said transcribed nucleic acid sequences generated by the library from which said contigs are derived.

According to still further features in the described preferred embodiments, each of the plurality of the nucleic acid constructs further includes a coding nucleic acid sequence of a known protein covalently linked to the specific non-coding nucleic acid sequence.

According to still further features in the described preferred embodiments, the at least two distinct organisms represent closely related species.

According to still further features in the described preferred embodiments, isolating the non-coding nucleic acid sequences is effected by: (i) computationally clustering transcribed nucleic acid sequences of the organism to thereby obtain a plurality of clusters; (ii) computationally generating contigs from at least a subset of the plurality of clusters; (iii) computationally aligning the contigs with the genomic nucleic acid sequences of the organism to thereby identify inter-contig region sequences of the genome of the organism; and (iv) amplifying the inter-contig region sequences to thereby obtain the isolated non-coding nucleic acid sequences.

According to still further features in the described preferred embodiments, the method of uncovering regulatory sequences functional in a biological pathway of an organism, further comprising assigning to the contigs a score according to at least one parameter selected from the group consisting of: (a) the number of the transcribed nucleic acid sequences clustered; (b) the percent homology of nucleotide sequences of the contigs to nucleotide sequences of known transcription factors; (c) the percent homology of nucleotide sequences of the contigs to nucleotide sequences of selected genes; (d) the number of expression libraries from which the contigs were generated; (e) the number of types of expression libraries from which the contigs were generated; (f) the number of RNAs comprised in the plurality of clusters; (g) the length of the contig; (h) the types of methods whereby the transcribed nucleic acid sequences were derived; (i) the type of tissues from which the transcribed nucleic acid sequences were derived; (j) the developmental stage of the tissues from which said transcribed nucleic acid sequences were derived (k) the growth conditions of the tissue from which said transcribed nucleic acid sequences were derived; and (l) the number of clusters of said transcribed nucleic acid sequences generated by the library from which said contigs are derived.

According to still further features in the described preferred embodiments, the method of generating phenotypic variation in an organism further comprising the step of culturing the plurality of organisms genetically transformed with the non-coding nucleic acid sequences under conditions suitable for identifying the phenotypic variation.

According to still further features in the described preferred embodiments, the method of generating phenotypic variation in an organism, further comprising assigning to the contigs a score according to at least one parameter selected from the group consisting of: (a) the number of the transcribed nucleic acid sequences clustered; (b) the percent homology of nucleotide sequences of the contigs to nucleotide sequences of known transcription factors; (c) the percent homology of nucleotide sequences of the contigs to nucleotide sequences of selected genes; (d) the number of expression libraries from which the contigs were generated; (e) the number of types of expression libraries from which the contigs were generated; (f) the number of RNAs comprised in the plurality of clusters; (g) the length of the contig; (h) the types of methods whereby the transcribed nucleic acid sequences were derived; (i) the type of tissues from which the transcribed nucleic acid sequences were derived; (j) the developmental stage of the tissues from which the transcribed nucleic acid sequences were derived; and (j) the developmental stage of the tissues from which said transcribed nucleic acid sequences were derived (k) the growth conditions of the tissue from which said transcribed nucleic acid sequences were derived; and (l) the number of clusters of said transcribed nucleic acid sequences generated by the library from which said contigs are derived.

According to still further features in the described preferred embodiments, the method of generating phenotypic variation in an organism, further comprising covalently linking a coding sequence of a known protein to each of the non-coding nucleic acid sequences prior to step (b).

According to still further features in the described preferred embodiments, the method of generating phenotypic variation in an organism, further comprising generating a plurality of organisms genetically transformed with the non-coding nucleic acid sequences, isolating a non-coding nucleic acid sequence from each organism which exhibits phenotypic variation and using isolated non-coding nucleic acid sequences for the combinatorial shuffling of step (b).

According to still further features in the described preferred embodiments, the method of generating phenotypic variation in an organism, further comprising characterizing the non-coding nucleic acid sequences prior to step (b).

The present invention successfully addresses the shortcomings of the presently known configurations by providing methods platforms and kits for identifying and isolating regulatory sequences of an organism and for using such regulatory sequences to generate genotypic and optionally phenotypic variation in the organism. In addition, the present invention can also be utilized to identify non-coding sequences which regulate an expression of genes functional in biological pathways

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a flow-chart depicting steps in the process of bioinformatic identification of candidate DREs from DNA sequence databases.

FIGS. 1b-c are schematic diagrams depicting cloning of candidate DREs via PCR-amplification from fully sequenced genomic DNA (FIG. 1b) and from ESTs (FIG. 1c) into expression vectors. Red arrows indicate external and nested primers for PCR amplification.

FIGS. 2a-c are schematic diagrams depicting strategies of the present invention for identification and PCR cloning of unidirectional candidate DREs located within inter-contig region sequences 0.2-6 kb and>6 kb in length, and of bidirectional DREs located within inter-contig region sequences 0.2-6 kb in length (FIGS. 2a, 2b and 2c, respectively). Contig-defined sequence (CDS) transcription putatively regulated by amplified DREs, and the directionality thereof, are indicated by bent arrows.

FIG. 3 is a data plot depicting numbers of unidirectional (navy lozenges) and bidirectional (pink squares) candidate DREs (≦6 kb in length) versus length of candidate DREs. The y-axis numbers are only directly applicable for the unidirectional candidate DREs. For bidirectional candidate DREs, the y-axis represents the proportion of these DREs in the entire candidate DRE population.

FIG. 4 is a data plot depicting numbers of unidirectional (navy lozenges; left-hand side y-axis units) and bidirectional (pink squares; right-hand side y-axis units) candidate DRE regions (100-1,500 bp) versus candidate DRE length.

FIG. 5 is a composite agarose gel photograph/schematic diagram depicting a scheme for cloning of vectors for expression of luciferase reporter genes under the control of candidate DREs.

FIG. 6 is a schematic diagram depicting generation of novel genotypes via combinatorial shuffling of heterologous DRE/expressed sequence pairs within a given genome.

FIG. 7 is a data histogram depicting the percentages of “True”, “False” and “Mixed” candidate DREs out of: all inter-contig regions initially identified (dark blue columns, n=16,176); the inter-contig regions remaining after discarding those not located upstream of contigs, those located upstream of contigs but not comprising at least one RNA sequence and those assembled from<4 ESTs (dark red columns, n=4,588); and the inter-contig regions having one (yellow columns) or both (light blue columns) flanking contigs whose sequence(s) correspond(s) to known RNA sequence(s).

FIG. 8 is a data histogram depicting percentages of “True”, “False” and “Mixed” DREs which were identified in a search for candidate DREs matching selected biological criteria.

### DESCRIPTION OF THE PREFERRED EMBODIMENTS

The present invention can be used to (i) isolate non-coding sequences from a genome of an organism, (ii) uncover non-coding sequences participating in biological pathways and (iii) generate genotypic, and optionally phenotypic, variation in an organism.

The principles and operation of the present invention may be better understood with reference to the accompanying descriptions.

Before explaining at least one embodiment of the invention in detail, it is to be understood that the invention is not limited in its application to the details of construction and the arrangement of the components set forth in the following descriptions or illustrated in the Examples section. The invention is capable of other embodiments or of being practiced or carried out in various ways. Also, it is to be understood that the phraseology and terminology employed herein is for the purpose of description and should not be regarded as limiting.

The phenotype of an organism is dictated by a combination of the amino acid sequences of its gene products and the spatial/temporal expression patterns thereof.

Such expression patterns are regulated by DNA regulatory elements (DREs) which are located within non-coding regions of the genome.

As used herein, a “non-coding” region is a non-transcribed polynucleotide.

As such, isolation and characterization of such non-coding regions can provide insight into the regulatory mechanisms underlying phenotypic variation.

Examples of DREs are well known to those of ordinary skill in the art to include, for example, promoters, enhancers, suppressors, silencers, locus control regions and the like.

It will be understood by one of ordinary skill in the art that isolation of DNA regions or sequences can be effectively achieved by cloning of such regions in, for example, plasmid vectors.

Since non-coding regions comprise the majority of the DNA sequences of genomes of organisms, isolation and characterization thereof can prove to be a time consuming task.

Thus, according to one aspect of the present invention there is provided a computing platform which can be utilized to identify non-coding sequences, such as, for example, inter-contig region sequences of an organism and to generate primer sequences for amplifying such sequences, thus enabling the rapid and efficient cloning of such sequences from an organism.

The computing platform includes a processing unit which operates a software application designed for: (a) generating data pertaining to inter-contig sequences of the organism; (b) processing data and classifying sequences according to various biological parameters (see Example 2 of the Examples section) and (c) automatically generating primer sequences suitable for amplifying the inter-contig sequences of the organism.

Preferably, the data pertaining to inter-contig sequences of the organism is generated by clustering transcribed nucleic acid sequences of an organism to thereby generate contigs (for further description of clustering and contig generation, see the Examples section which follows).

As used herein, the phrase “transcribed nucleic acid sequences” refers to nucleic acid sequences being, or corresponding to, gDNA sequences, or gDNA sequences having the capacity to be transcribed, from gDNA into RNA. It will be appreciated that genomic sequences may comprise intronic sequences being absent from corresponding transcribed nucleic acid sequences as a result of, for example, intron splicing during RNA processing.

Examples of transcribed nucleic acid sequences include unspliced RNA sequences, such as, for example, primary RNA transcript sequences; spliced RNA sequences, such as, for example, mRNA sequences; poly-adenylated (polyA) RNA sequences; expressed sequence tags (ESTs); cDNA sequences, computationally identified nucleic acid sequences, and the like.

It will be appreciated that nucleic acid sequences having the capacity to be transcribed, from gDNA into RNA can be identified by computational analysis using techniques well known to one of ordinary skill in the art.

As used herein, a “contig” is defined as a polynucleotide having a sequence being, or essentially corresponding to, a gDNA sequence represented by a cluster of transcribed nucleic acid sequences, as described in the Examples section which follows. It will be appreciated that gDNA sequences corresponding to a contig may comprise intronic sequences being absent from one or more transcribed sequences clustered to generate the contig, in cases, for example, where such transcribed nucleic acid sequences are obtained, either directly or indirectly from RNA sequences from which introns have been spliced out. such as, for example, cDNA sequences, as described in the Examples section which follows.

As used herein, the phrase “inter-contig region” refers to a nucleic acid sequence being, or corresponding to, a gDNA sequence located between consecutive contigs.

In cases wherein EST sequences derived from an EST library of an organism are clustered, the computing platform can also be used to determine if the size of the EST library utilized is sufficient for constructing substantially all of the coding sequences of the organism's genome.

This is effected by first predicting the number of genes present in the genome of the organism (N) followed by obtaining a product of N(ln(N)+C), wherein C=0.5772. The product of this equation represents the minimal number of ESTs needed for constructing substantially all of the coding sequences of the genome of the organism (for a more detailed description of this process, see the Examples section which follows).

Preferably, expressed sequences, such as ESTs, are obtained from as many different types of libraries as possible, such as, for example, libraries from different laboratories, libraries from different tissue types, libraries from tissues growing under different growth condition and libraries in which expressed sequences were synthesized from both transcriptional orientations. Such variation in libraries can be used to optimize correlation of number of expressed sequences with the size of the clusters generated therewith as well as representation of the 5′ region.

Preferably, libraries comprising at least 100 expressed sequences are employed. Following clustering, the contigs generated are aligned with genomic sequences of the organism, thus defining the inter-contig regions of the genome.

Following definition of the inter-contig regions, primer sequences suitable for amplifying the inter-contig sequences of the organism are then automatically provided by the computing platform according to the sequences of the inter-contig regions defined thereby.

In the absence of genomic information, the 5′ and 3′ sequences of the generated contigs can be utilized to generate primers suitable for amplifying the inter-contig sequences of the organism. In such cases, the 5′ portion of a contig can be used to define a primer sequence useful for directing amplification of upstream sequences, while the 3′ portion can be utilized to define primer sequences useful for directing amplification of downstream sequences.

Although the latter approach is inherently limited by the lack of information needed for contig alignment, and thus 5′ and 3′ primer matching, this limitation can be overcome by pairing a 5′ or a 3′ primer with a degenerate or universal primer, or by using a mixed pool of 5′ and 3′ primers in a single PCR reaction or in several PCR reactions using varying primer combinations. In such cases, it is preferable that amplification conditions are selected so as to optimize primer-template specificity, thus ensuring that the PCR products generated are indeed amplified inter-contig regions.

The latter approach can be substantially improved by using primers derived from contigs representing syntenic, or homologous, chromosomal sequences of a related organism. Thus, extension of clusters of an Arabidopsis related plant, such as tomato, can be facilitated using primers derived from syntenic or homologous Arabidopsis clusters. Methods of assigning synteny, or homology, to chromosomal sequences of a pair of related organisms are well known to those versed in the art. For example, assignment of chromosomal synteny or homology between chromosomes from different genomes can be derived computationally by comparing batches of mRNA derived sequences derived from two species, such as Arabidopsis and tomato, using the TBLASTX method, as described in the Examples section which follows.

Further descriptions of the above approaches and additional approaches for high throughput identification and isolation of inter-contig regions are provided in the Examples section which follows.

In addition to the above, the inter-contig regions of an organism identified according to the teachings of the present invention can be sequenced and the sequence information stored as a database on a computer readable media (see the Examples section below for further detail). Such database information can be analyzed to uncover consensus sequences, non-redundant sequences or any other sequence characteristic which can provide information as to the function of the inter-contig region. Comparison of two or more databases of related or non-related species can also be effected in efforts to uncover yet additional information.

Since inter-contig regions define non-transcribed regions of a genome, inter-contig regions, according to the method of the present invention comprise DREs.

Methods of determining whether, and/or with what relative probability, an inter-contig region comprises a DRE are set forth in the Examples section which follows.

As shown in the Examples section which follows, the present invention provides for a wide variety of means whereby contigs having desired characteristics can be identified or selected from databases.

In order to select contigs representing with maximal accuracy and/or completeness expressed sequences, contigs generated by clustering the largest number of transcribed nucleic acid sequences are selected. Preferably, contigs are assembled from at least 4 transcribed nucleic acid sequences are selected. According to another embodiment, contigs generated from clusters comprising the largest possible number of RNA sequences are selected. Preferably, contigs are generated from clusters comprising at least two RNA sequences are selected. Preferably, contigs from libraries expressing the largest possible number of contigs, preferably no less than 100, are selected so as to maximize the probability of selecting contigs representing with maximal accuracy and/or completeness expressed sequences. According to yet another embodiment selection of such contigs is preferably effected by selecting contigs defined by clustered transcribed nucleic acid sequences generated by the largest possible number of different laboratories, techniques, etc. For example, contigs generated from libraries of expressed sequences transcribed from both coding strands are selected.

Selection of contigs having maximal probability of representing a complete transcript is preferably effected by selecting contigs whose length is between 0.5 and 6 kb, more preferably between 1 and 3 kb.

In order to maximize the probability of selecting contigs representing a given type of protein, such as a TF, contigs having the highest possible level of nucleic acid sequence homology to genes encoding such proteins, as determined, for example, by homology searches of annotated databases, are selected. Preferably homology searches for contigs representing proteins, such as TFs, using the GO database are performed with a cut-off e-score no larger than 10⁻⁴. Preferably homology searches using the Pfam database for identifying contigs comprising domains, such as TF domains, are effected using a SCORE cut-off threshold of at least 30.

Preferably selection of contigs representing constitutively expressed sequences is effected by selecting contigs which are represented by the largest possible number of expressed sequence libraries, by selecting contigs which are represented by the largest possible number of different types of expressed sequence, or preferably both.

In order to select contigs being specific to a given tissue type, developmental stage and/or growth condition, contigs generated using expressed nucleic acid sequence libraries derived from tissues of such a type, developmental stage and/or from tissues growing under such growth conditions.

According to a preferred embodiment of the present invention, selection or identification of DREs or non-coding sequences having desired regulatory properties is effected by selecting DREs defined by contigs displaying expression patterns characteristic of such regulatory properties, as demonstrated in the Examples section, below.

The non-coding sequences identified and/or cloned according to the teachings of the present invention can be utilized in several ways.

According to another aspect of the present invention there is provided a method of generating genotypic, and possibly phenotypic, variation in an organism. The method according to this aspect of the present invention is effected by isolating at least one non-coding nucleic acid sequence from a genome of the organism and genetically transforming the organism with the isolated sequence to thereby generate genotypic, and possibly phenotypic, variation in the organism.

Thus, the method according. to this aspect of the present invention alters gene expression by “repositioning” non-coding nucleic acid sequence(s) within a genome of the organism. It will be appreciated that placing gene coding sequences within regulatory control of heterologous elements present within the non-coding nucleic acid sequence, which elements do not regulate the expression of this gene in the wild type organism, may alter the expression pattern of this gene, thereby, for example, causing the activation of a dormant gene, or leading to altered metabolic, biochemical or biological pathways, all of which are capable of generating phenotypic variation in the organism.

The above described method is particularly useful for generating genotypic, and possibly phenotypic, variation in plants, in which such variations can provide commercially important traits.

It will be appreciated that various regions derived from non-coding nucleic acid sequences functional in altering a phenotype or phenotypes can be used to engineer “Mixed” non-coding nucleic acid sequences including regions of several related or unrelated non-coding nucleic acid sequences.

Such combinatorial shuffling of portions of non-coding nucleic acid sequences can be used to further increase their effect on a phenotype or phenotypes while also being useful in characterizing sequence “modules” which participate or contribute to the overall phenotypic effect of a particular non-coding nucleic acid sequence(s).

In order to identify commercially important traits in plants, the method of the present invention is preferably effected on a large scale using a plurality of plants and a plurality of isolated non-coding sequences.

Since in most cases phenotypic variation will lead to readily qualifiable/quantifiable traits, the method of generating phenotypic variation according to the teachings of the present invention can employ high throughput approaches. For example, the isolated sequences (e.g., DREs) of the present invention can be generated, cloned and introduced into an organism (e.g., a plant) using a “one tube” approach.

In such an approach, a single reaction tube can be used to PCR amplify the DRE(s) of interest using specific primers or primers sets; to enzymatically digest PCR products, if necessary; to clone such digested PCR products into a suitable propagation/transformation vector (as described hereinbelow); and to directly transform an organism therewith. Such a “one-tube” approach enables the automation of the method of the present invention, thus enabling large scale screening of numerous DREs. It will be appreciated that although a single tube approach typically reduces the efficiency of the digestion and/or cloning and/or propagation process(es), such reduction in efficiency is of no consequence in this case since most of the DREs used are typically of the same size and/or nucleotide complexity and, as such, no particular subset of DREs will be favored. In addition, the large scale applicability of the one-tube approach more than compensates for the reduced efficiency thereof. Example 7 of the Examples section which follows provides a more detailed description of the one-tube approach according to the teachings of the present invention.

To facilitate genetic transformation, the isolated sequences are preferably cloned into a nucleic acid construct.

Such a nucleic acid construct preferably further includes additional polynucleotide regions which provide a broad host range prokaryote replication origin and a prokaryote selectable marker. Where the heterologous sequence is not readily amenable to detection, the construct will preferably also have a selectable marker gene suitable for determining if a plant cell has been transformed. A general review of suitable markers is found in Wilmink and Dons, Plant Mol. Biol. Reptr. (1993) 11:165-185.

Suitable prokaryote selectable markers include genes conferring resistance to antibiotics such as ampicillin, kanamycin or tetracycline. Other polynucleotide sequences providing additional functions may also be present in the nucleic acid construct, as is known in the art.

Sequences suitable for permitting or enhancing integration of the polynucleotide sequence of the present invention into the plant genome are also recommended. These might include transposon sequences as well as Ti sequences which permit random insertion of a heterologous expression cassette into a plant genome.

Examples of nucleic acid constructs suitable for use by the present invention are provided in the Examples section which follows.

There are various methods of introducing nucleic acid constructs into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

Two main approaches which can be used to achieve stable integration of exogenous DNA into plant genomic DNA include:

(i) Agrobacterium mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fiber or silicon carbide whisker mediated transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues. In glass fibers or silicon carbide whisker mediated transformation, glass fibers or silicon carbide needles like structures are mixed with DNA and cells in a suspension to thereby induce fiber/whisker-cell collisions, which lead to cell impalement (by the fibers/whiskers) and DNA injection into the cell.

The transformation methods described hereinabove are typically followed by propagation of transformed tissues. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Due to the large number of non-coding sequences present in a plant genome, the method according to this aspect of the present invention is preferably effected on a large scale using a plurality of plants, each transformed with a specific nucleic acid construct harboring a specific non-coding sequence.

The resultant transformants can then be tested for general phenotypic variation detected by visible morphological alterations. Alternatively specific variations such as increased or acquired stress tolerance, and the like can be detected by cultivating the transformed plants under conditions suitable for detecting such phenotypes.

In any case, non-coding sequences of plants exhibiting phenotypic variation can be isolated using, for example, construct specific primers and the isolated sequence can be further characterized.

Although transformed plants which do not exhibit phenotypic variation are not readily utilizable, such plants can be genetically crossed with either wild type (w.t.) plants or closely related plant species in to generate progeny exhibiting phenotypic variation.

To increase genetic variation, the nucleic acid constructs of the present invention can also include a coding sequence of a characterized gene positioned under the transcriptional control of the non-coding sequence.

Such a characterized gene can encode, for example, diacylglycerol acyltransferase (Jako C. et al. Plant Physiol. 2001, 126(2):861-74), ATHB-8 HD-zip protein (Baima S. et al. Plant Physiol. 2001, 126(2):643-55), Leafy or Apetalal (Pena L. et al. Nat Biotechnol. 2001, 19(3):263-7), bacterio-opsin (Rizhsky L. and Mittler R. (Plant Mol Biol. 2001, 46(3):313-23), AtMYB23 (Kirik V. et al. Dev Biol. 2001, 235(2):366-77), cytokinin (Werner T. et al. Proc Natl Acad Sci USA. 2001, 98(18):10487-92) or any other gene capable of generating phenotypic variation when introduced into the plant under the transcriptional control of the non-coding sequence.

Positioning a coding sequence under the regulatory control of a non-coding sequence, such as an inter-contig region sequence, can also be utilized to uncover novel gene expression regulatory sequences and to identify regulatory sequences which regulate the expression of genes participating in biological pathways.

Thus, according to another aspect of the present invention there is provided, a method of identifying novel gene expression regulatory sequences. The method is effected by transforming an organism with an expression cassette including a non-coding nucleic acid sequence covalently linked to a reporter nucleic acid sequence, encoding, for example, a fluorophore, such as green fluorescent protein (GFP) or a derivative thereof, or an enzyme capable of catalyzing reporter activity, such as, for example β-galactosidase. The method is further effected by monitoring reporter activity, the reporter activity indicating a presence of a regulatory sequence in the non-coding nucleic acid sequence.

To uncover regulatory elements such as suppressors or enhancers, the expression cassette can further include a constitutive promoter sequence which can be positioned, for example, between the non-coding sequence and the reporter nucleic acid sequence.

Although the method according to this aspect of the present invention preferably employs a stable transformation approach, transient transformation of, specific plant tissues such as, for example, flower tissue, leaf tissue, seeds or tubers, which can be utilized for identifying tissue specific regulatory sequences or transient transformation of the whole plant can also be utilized.

In a stable transformation approach (described hereinabove), the expression cassette of the present invention is integrated into the plant genome and as such it represents a stable and inherited trait. In a transient transformation approach, the expression cassette of the present invention is expressed by the cell transformed but it is not integrated into the genome and as such it represents a transient trait.

Transient transformation can be effected by any of the direct DNA transfer methods described above or by viral infection using modified plant DNA viruses.

Transformation of an organism such as a plant with the expression cassette described above can also be utilized to identify regulatory sequences which regulate the expression of genes participating in specific biological pathways.

According to this aspect of the present invention, plants transformed with the expression cassette of the present invention are grown under conditions suitable for the induction of an uncharacterized biological pathway or are specifically stimulated by an agent capable of triggering the biological pathway.

If the non-coding region of the expression cassette includes a regulatory sequence (e.g. promoter) which participates in the biological pathway, then reporter activity is generated. In order to identify, and therefore discount, reporter activity which is unrelated to pathway induction, suitable controls, such as identical transformants grown under non-inducing conditions must be employed.

Following transformation and pathway triggering, plants exhibiting induced reporter activity are identified and the non-coding sequences used to effect the transformations are isolated therefrom, as described hereinabove. The isolated sequence can then be utilized to further characterize the regulatory sequence and/or to isolate and characterize the coding sequence naturally regulated by this regulatory sequence in the genome of the organism.

Thus, the present invention provides methods of identifying and isolating non-coding sequences present in, for example, inter-contig regions. In addition, the present invention also provides methods of utilizing isolated non-coding sequences for generating genotypic and possibly phenotypic variation as well as for uncovering novel regulatory sequences and regulatory sequences participating in specific biological pathways.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes 1-111 Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Databases of Transcribed Nucleic Acid Sequences of the Arabidopsis Genome

Databases of the nucleotide sequences of the transcribed and non-transcribed regions of the genomes of organisms, such as the heavily investigated model plant Arabidopsis, would be of great value in industries and fields employing plants or plant products. Such databases would constitute a powerful bioinformatics tool for the processing of information regarding Arabidopsis transcribed nucleic acid sequences and for applied uses thereof. Furthermore, databases of sequences of transcribed regions of the genome would enable creation of databases of sequences of non-transcribed regions of the genome comprising DREs. The latter type of database could be used to efficiently isolate and clone DREs which could be used to produce genetically modified plants exhibiting desired characteristics. Such databases, however, are currently lacking. Thus, the present inventors have created databases of transcribed nucleic acid sequences of the Arabidopsis genome, as described below.

Materials and Methods:

Identification of Transcribed Regions of the Arabidopsis Genome:

The longest possible stretches of transcribed Arabidopsis sequences were computationally identified using sequencing data from mRNA and expressed sequence tag (EST) databases containing≧10⁵ sequences. This included sequences stored in GenBank which, at the time of writing, included 115.4 Mb of the 125 Mb Arabidopsis genome (The Arabidopsis Genome Initiative, Nature 2000, 408:796), data from EST databases containing approximately 125,000 ESTs from 48 libraries and data from 997 well-characterized genes from the SwissProt database. Transcribed nucleic acid sequences were computationally clustered and assembled to create contigs defining the maximal contiguous stretches of transcribed nucleic acid sequences using LEADS™ software (Compugen). This software also recognizes vector sequences used for producing the ESTs, sequencing quality, frequency of low-complexity regions, repetitive sequences within transcribed regions, cases and frequencies of alternative splicing and intron retention and cases and frequencies of antisense RNAs.

Mathematical Prediction of Minimal EST Database Size Required to Represent All of the Genes in a Genome with High Probability:

The minimum number of EST entries in a database required to represent all of the genes in a genome is within the range calculated by the following equations: n×(1n(n)+ₓ) and 2×n**(1n(n)+ₓ), where n=number of contigs (predicted number of genes in the genome) and ₓ about 0.5772. These equations are based on the assumption that ESTs are produced from samples qualitatively representing all the genes of the particular genome. For example, if it is assumed that the Arabidopsis genome comprises 25,000 genes, then the minimal Arabidopsis EST library containing>1 EST/gene should contain between 267,596 and 535,192 EST entries.

Results:

Computational Identification of Transcribed Nucleic Acid Sequences:

Processing of Arabidopsis sequence databases using LEADS™ software generated 19,311 contigs based on clustering and assembly of EST and RNA sequences. This number is less than the 25,498 genes identified by the Arabidopsis genome initiative (The Arabidopsis Genome Initiative, Nature 2000, 408:796). These differences are due to the fact that the LEADS™ algorithm generated contigs through a process of EST and RNA sequence clustering and assembly, whereas only 61% of the genes generated by the Arabidopsis genome initiative are products of such a process. An analysis of the distibution of contig number vs. number of clustered sequences per contig in the data set employed to identify transcribed nucleic acid sequences is shown in Table 1.

TABLE 1
Distibution of contig number vs. number of clustered
sequences per contig in a data set employed to
identify transcribed nucleic acid sequences.

| | No. of contigs composed | |
|---|---|---|
| No. of clustered | of “X” number | Fraction of |
| sequences/contig (= “X”) | of sequences | total contigs (%) |
| 2 | 6,406 | 33 |
| 3 | 4,383 | 23 |
| 4 | 2,556 | 13 |
| 5 | 1,514 | 8 |
| 6 | 935 | 5 |
| 7-10 | 1,790 | 9 |
| 11-20 | 1,117 | 6 |
| 21-99 | 579 | 3 |
| 101-435 | 31 | 0.16 |
| Total: | 19,311 | |

Detailed LEADS™ analysis revealed that 56% of the contigs were composed of either single clusters uniquely defining 1 mRNA sequence and/or were defined by>3 clustered ESTs sequences. Such contigs were not expected to reliably represent complete transcribed gene sequences and were discarded. Contigs composed of at least 4 ESTs or RNA sequences were found to represent 46% of the contig population. Contigs belonging to this group have a high probability of accurately representing transcribed gene sequences. Numbers of clustered ESTs defining contigs were correlated with contig expression levels and the probability that contigs accurately represent entire transcribed nucleic acid sequences. Numbers of overlapping contigs defined by different expressed sequence clusters were correlated with the probability of alternative splicing, inaccurate clustering or incomplete representation of expressed sequences by EST clusters.

An analysis of the distribution of a population of 19,311 contigs with respect to contig length, as obtained using the LEADS™ program, is shown in Table 2. The observation that 91% of the contigs were between 1-3 kb in length, and therefore fall within the expected size range for genes, provides evidence that the contigs generated with the LEADS™ algorithm correctly represent transcribed gene sequences.

TABLE 2
Distribution of contig lengths.

| Contig length | | Fraction of total |
|---|---|---|
| (bp) | No. of contigs | (%) |
| <1,000 | 50 | 0.25 |
| 1,000-1,999 | 13,798 | 72 |
| 2,000-2,999 | 3,734 | 19 |
| 3,000-3,999 | 1,029 | 5.4 |
| 4,000-5,999 | 511 | 2.4 |
| 6,000-10,000 | 153 | 0.8 |
| >10,000 | 36 | 0.2 |
| Total: | 19,311 | |

These results therefore indicate that the expressed sequence databases of the present invention have the capacity to effectively and accurately provide information regarding such sequences on a genome-wide scale. As such, these databases constitute a potent bioinformatics tool applicable in industries or fields employing plants or plant products and which can furthermore be employed to create genome-scale databases of sequences of DRE comprising regions of the Arabidopsis genome.

### Example 2

### Creation of an Arabidopsis Expressed Sequence Database Searchable According to Sets of Prioritizable Biological Parameters

Information correlating the genomic sequence of genes of the model plant Arabidopsis with biological characteristics thereof, such as expression profiles, is highly desirable since such information provides a powerful bioinformatics tool which can be exploited, for example, in industries and fields employing plants and plant products. To date, however, only a small fraction of Arabidopsis genes have been characterized with regards to biological parameters, such as, for example, expression profiles. In order to provide such information, the present inventors have created a database of sequences of expressed regions of the Arabidopsis genome annotated and searchable according to sets of prioritizable biological parameters, as follows.

Materials and Methods:

EST Library Selection for Generation of an EST Database Annotated According to Relevance with Respect to Biological Parameters:

Out of 458 available EST libraries, 48 containing>50 ESTs were selected for generation of an EST database annotated according to sets of prioritizable biological parameters. The libraries selected were derived from sources representing various combinations of anatomical locations and sublocations, developmental stages and growth treatments, as shown in Table 3.

Annotation of ESTs According to Sets of Prioritizable Biological Parameters.

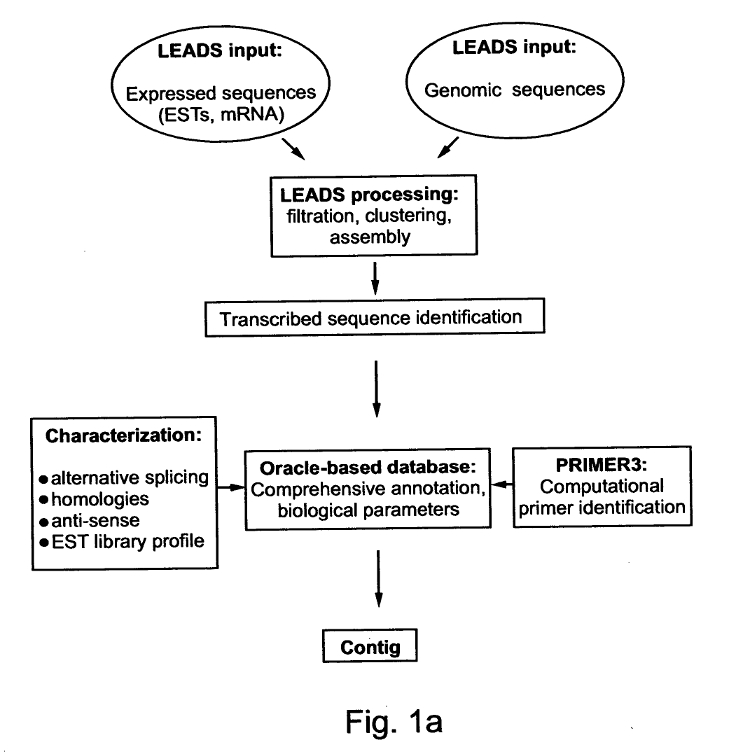

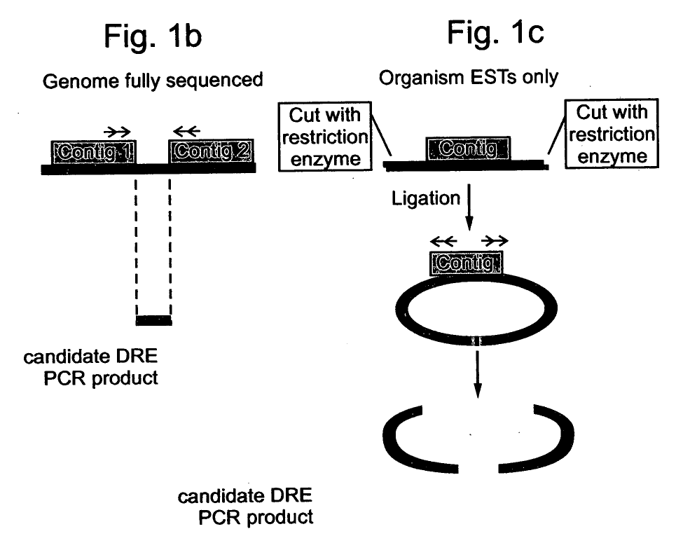

In order to enable database query output in the form of contigs prioritized according to sets of biological parameters, the EST sequence database described above was annotated by assigning to each EST a score on a scale of 1-100 with respect to the biological parameters (sub-groups) described in Table 3. The information contained in Table 3 was integrated into an Oracle-based database enabling parameter-prioritized querying. The process of generating such a database is schematized in FIG. 1a.

The successful functioning of this system depends on several factors, including; careful assignment of relevance with respect to biological parameters, sufficient volumes of data, sufficient computer processing power, suitable data mining tools and effective manual feedback in response to computer-generated output.

TABLE 3
EST libraries utilized to generate an EST sequence database
annotated according to relevance with respect to biological parameters.

| | | | | Fraction of |
|---|---|---|---|---|
| EST library | No. of | | | total ESTs |
| tissue name | libraries | Library sub-group | No. of ESTs | (%) |
| Seedling | 10 | | 6,834 | 3.94 |
| | 1 | Whole seedlings, 10-14 days old | 2,322 | 1.34 |
| | 4 | Hypocotyls, 3 days old | 2,050 | 1.18 |
| | 2 | in vitro grown, etiolated, 5 days old | 1,645 | 0.95 |
| | 3 | Subtracted library from NaCl treated whole plants | 817 | 0.47 |
| Flower | 7 | | 14,035 | 8.08 |
| | 2 | Flower | 12,639 | 7.28 |
| | 1 | Flower tips | 116 | 0.07 |
| | 3 | Inflorescence | 1,260 | 0.73 |
| | 1 | Flower-display | 20 | 0.01 |
| Seed | 6 | | 32,279 | 18.59 |
| | 2 | Seeds | 11,094 | 6.39 |
| | 3 | Green siliques | 26,185 | 15.08 |
| Root | 5 | | 36,927 | 21.27 |
| | 3 | roots, untreated, 4-7 weeks old | 36,656 | 21.11 |
| | 1 | roots, nitrate treated | 217 | 0.12 |
| | 1 | roots, nematode infested | 54 | 0.03 |
| Leaf | 9 | | 18,289 | 10.53 |
| | 5 | mature leaf | 15,613 | 8.99 |
| | 3 | rosette | 2,351 | 1.35 |
| | 1 | green shoot | 325 | 0.19 |
| Cell culture | 5 | | 1,178 | 0.68 |
| | 3 | cell cycle | 1,178 | 0.68 |
| Above- | 7 | above-ground organs | 64,072 | 36.9 |
| ground | | | | |
| organs | | | | |
| Total: | 49 | | 173,614 | 100 |

Algorithms Employed to Generate an Annotated EST Database Query Output in the Form of Contigs Prioritized According to Sets of Biological Parameters:

The following equations instruct the automatic and interactive prioritization of contigs based on a particular set of preferences:

Expression in a specific organ: If [Number of categories]=1, then Specific=[number of libraries]*X*Y, else Specific=0.

Contig quality: Reflects a user-defined priority list.

If [Subjective]≧90 then Mark=([Subjective interest of cluster]-89)*Y, else Mark=0.

Constitutive expression: Prioritizes contigs with constitutive expression. Numbers of clustered ESTs comprised in contigs and/or numbers of different libraries from which the same contigs are derived is/are correlated with the probability that such contigs are constitutively expressed

Constitutive=[number of categories]*Y.

Intermediate parameter X: If [Subjective contig interest]>50 (scale 1-100) then X=[Subjective contig interest] else, X=1.

Intermediate parameter Y: If [Number of ESTs]+[Number of mRNAs])≧200, then Y=100, else Y=([number of ESTs]+[Number of mRNAs])/2.

Assignment of TF Status to Contigs:

High priority is assigned to TF genes on the assumption that they are responsible for large fraction of genetic and phenotypic variation. Contigs were classified as TFs if their sequences were found to be homologous to sequences listed in the Arabidopsis Gene Ontology (GO), GenBank non-redundant (nr) or Pfam databases. Contigs were classified as ozone responsive TFs on the basis of information provided to the present inventors by Nina Fedoroff.

GO Analysis:

Contigs were assigned TF status by performing homology comparison with the GO annotated database with TBLASTX using a cutoff e-score threshold of 10⁻⁴. Contigs scoring very high homologies were manually confirmed as being TFs.

Pfam Analysis:

Homology searches using the Pfam protein domains database was used to identify contigs comprising TF domains. Homology searches using Pfam were performed using a SCORE cut-off threshold of≧30.

Experimental Results:

Distribution of Arabidopsis Contigs Prioritized According to Biological Criteria:

Analysis of the annotated EST database described above using the algorithms described above identified 544 contigs assigned high scores with respect to various biological parameters, as shown in Table 4. The percentage of ESTs unique to each parameter (subgroup) was found to vary from a low of 0.01% to a high of 4.1% (in mature leaf- and root-derived ESTs, respectively). The average fraction of ESTs unique to a single subgroup was calculated to be 1.3%, thus relatively few expressed genes have organ- and/or developmental stage-specific expression. Moreover, no differences were found in the percentages of ESTs uniquely expressed during specific developmental stages. For example, only 0.7% of ESTs were found to be specifically expressed in cycling cultured cells.

TABLE 4
Biological characterization of contigs with respect to tissue
specificity of expression and inducibility under set growth conditions.

| | | Contig/EST data | | |
|---|---|---|---|---|
| | | No. of | | Fraction organ |
| | Interest score | organ | Total no. of ESTs | specific ESTs |
| | manually assigned | specific | comprising organ | out of total no. |
| | to EST library | contigs in | specific contigs in | ESTs in library |
| EST library* | (%) | library | library | (%) |
| flower buds | 100 | 25 | 71 | 0.6 |
| seeds, 3-15 days | 100 | 70 | 329 | 3 |
| post-flowering | | | | |
| cultured cycling cells | 100 | 7 | 8 | 0.7 |
| cycling cells inoculated with | 100 | 2 | 2 | |
| Xanthomonas campestris | | | | |
| strain 147 | | | | |
| etiolated seedlings | 95 | 9 | 16 | 1 |
| subtracted library from | 95 | 1 | 1 | 1 |
| NaCl-treated seedlings | | | | |
| green siliques | 90 | 44 | 231 | 0.9 |
| dry seeds | 70 | 5 | 12 | 3.5 |
| roots | 60 | 12 | 150 | 4.1 |
| leaves | 20 | 2 | 2 | 0.01 |
| above-ground organs | 10 | 174 | 861 | 1.35 |

Three types of tissues were found to contain a relatively high percentage of specifically expressed contigs (i.e., genes): roots (4.1%), developing seeds (3%) and dry seeds (3.5%). The high proportions of unique ESTs found in roots and seeds could be due to the fact that the plant organs used for the preparation of ESTs of these categories were absent from the biological material used to prepare the above-ground/whole plant EST libraries. ESTs belonging to the above-ground category include most of the other EST subgroups. Dry seed- and developing seed-derived ESTs were both found to express a high proportion of unique, developmental stage-specific sequences. Above-ground/whole plant-derived ESTs comprised 174 contigs containing 861 unique sequences, indicating that this subgroup included additional organs/tissues from other subgroups.

Contig Quality Classification:

345 contigs were found to have a quality score>1 and these comprised a high proportion of ESTs from subgroups (flower tips, cell culture, etiolated seedlings, seed embryos, NaCl-treated seedlings, nematode-infected roots and nitrate-treated roots) given an interest score≧90 out of 100.

Constitutively Expressed Genes:

159 contigs were classified as being highly constitutively expressed (score>500). For example, one of the highest scoring contigs was assembled from 736 ESTs derived from 14 different EST library subgroups. This sequence was found to have significant homology to that of the hsc70 gene of Lycopersicon esculantum and has been found to be highly expressed in the vegetative tissues of this plant (Sun S W. et al. 1996. Gene 170:237).

Transcription Factors:

Homology searches using GO database were used to assign putative TF status to 367 contigs, however this number was reduced to 59 contigs satisfying selection criteria. Homology searches using GenBank nr database were used to assign putative TF status to 472 contigs, with 285 of these meeting selection criteria. Homology searches using Pfam database was used to assign putative TF status to 798 contigs, however this number was reduced to 421 fulfilling selection criteria. On the basis of information provided to the present inventors by Nina Fedoroff, 4 contigs were assigned ozone induced TF status. In sum, 586 contigs satisfying filtration criteria out of a total of 1,460.

Contigs defined as TFs according to selection criteria were comprised, of no more than 7 clustered ESTs and were derived, on average, from no more than 3 EST libraries. Contigs defined as TFs which did not satisfy selection criteria were comprised, on average, of 1.8 clustered ESTs and were derived, on average, from 1.1 EST libraries. The average TF gene of both groups is composed, on average, of 4 clustered ESTs derived from at least 2 EST libraries.

The distribution of database specific annotation of TF contigs with a set of general biological parameters is shown in Table 5. In total 140 contigs assigned TF status were 100% specific to one library type and 48 of these were specific to a library whose type designation was manually selected as being useful for biological parameter annotation of contigs. The number of contigs annotated as novel, as defined by the absence of homologous expressed sequences in the databases searched, was 188, and the numbers of contigs defined as being constitutively and inducibly expressed were 22 and 6, respectively.

TABLE 5
Database specific annotation of TF contigs
with general biological parameters.

| | | Number of 100% library- | | Number of | Number of |
|---|---|---|---|---|---|
| | Number of 100% | specific contigs having an | Number | constitutively | inducibly |
| Annotation | library-specific | annotatable (organ-specific) | of novel | expressed | expressed |
| source | contigs | biological specificity | contigs* | contigs | contigs |
| GO | 26 | 13 | — | — | — |
| GenBank nr | 90 | 26 | 44 | 7 | 1 |
| Pfam | 24 | 9 | 144 | 15 | 1 |
| Federoff | — | — | — | — | 4 |
| (ozone) | | | | | |
| Total | 140 | 48 | 188 | 22 | 6 |

*without corresponding RNA sequence

A subgroup comprising 35% of the initial set of 335 contigs classified as TFs was classified as being organ specific in various organs (Table 6).

Based on genomic studies performed in nematode and in Drosophila, it is estimated at the time of writing that almost 10% of plant genes (about 1700 genes) function as TFs.

These results therefore demonstrate that the annotated EST databases of the present invention constitute a powerful bioinformatics tool for correlating gene sequences with biological characteristics thereof and furthermore enable generation of databases of non-transcribed regions of the Arabidopsis genome. As such, these databases provide information which can be effectively exploited in fields employing plants and plant products.

TABLE 6
Distribution of tissue-specificity of contigs classified as TFs.

| Tissue source of | Number of | |
|---|---|---|
| EST libraries* | contigs classified as TFs | Fraction of contigs (%) |
| rosette | 16 | 5 |
| roots | 27 | 8 |
| green siliques | 28 | 8 |
| leaf | 7 | 2 |
| flower | 5 | 1.5 |
| seed | 17 | 5 |
| seedling | 13 | 4 |
| inflorescence | 5 | 1.5 |

### Example 3

### Computational Identification of Arabidopsis Candidate DREs

Genome-scale databases of DRE nucleotide sequences of organisms, such as Arabidopsis, are highly desirable since these would constitute a valuable bioinformatics tool enabling analytical processing of DREs related information on a genomic scale. Furthermore, such databases could enable the efficient cloning of such DREs and these could be used in a multitude of applications, such as, for example, to generate plants genetically modified to possess novel and/or selected characteristics. Hence, the capacities afforded by such databases could clearly be exploited to great benefit in industries employing plants and plant products. To date, however, very little information regarding DREs of the model plant Arabidopsis, is available. Thus, in order to provide such information, the present inventors have generated genome-scale databases of Arabidopsis DREs, as follows.

Materials and Methods:

Computational Identification of Candidate DREs:

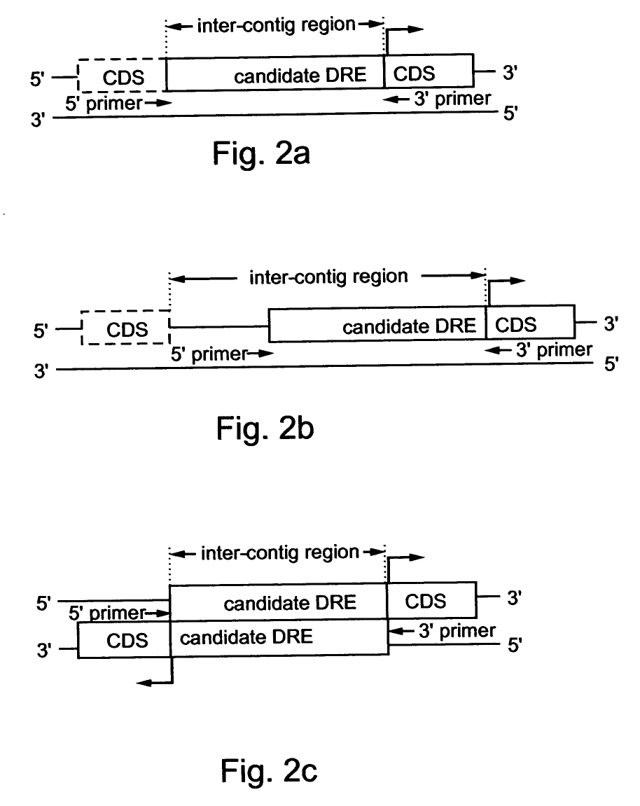

The Arabidopsis contig sequences stored in the contigs databases described above were aligned with available Arabidopsis gDNA sequences using LEADS™ software (Compugen) and sequences of gDNA corresponding to those of contigs (including gDNA sequences comprising intronic sequences absent from the corresponding contigs, in cases where such contig sequences were derived directly or indirectly from spliced RNA sequences) were defined as contig-defined sequences (CDSs). Regions of gDNA computationally determined to be positioned between adjacent CDSs were then classified as “inter-contig” regions. Inter-contig regions located within regions 6 kb upstream of CDSs were defined as candidate DREs for such regions, based on the fact that promoters are generally located within the region 6 kb upstream of the coding sequences which they regulate. It was demonstrated that the minimal length of DREs is>200 bp (see below), therefore no sequences≦200 bp in length were classified as candidate DREs. Strategies for PCR cloning of candidate DREs from fully sequenced gDNA and from ESTs are shown in FIGS. 1b and 1c, respectively. Strategies for identification and PCR cloning of unidirectional candidate DREs located within inter-contig region sequences 0.2-6 kb and>6 kb in length, and of bidirectional DREs located within inter-contig region sequences 0.2-6 kb in length are shown in FIGS. 2a, 2b and 2c, respectively. In cases where inter-contig region nucleic acid sequences were not available, inter-contig sequences extending 3-5 kb upstream of the contig were cloned by PCR and inverse PCR amplification of DNA from a gDNA library and from whole gDNA, respectively. Such cloned inter-contig regions were sequenced and the data were added to a computational DRE database.

The candidate Arabidopsis DRE population in the database thereby generated was analyzed with respect to directionality- and length-vs-frequency distribution.

Results:

Size and Orientation Profile of Computationally-Identified Arabidopsis Candidate DREs and their Genomic Frequency:

The total number of identified inter-contig regions was 16,176. The number of inter-contig regions comprising candidate DREs located upstream of contigs assembled from clusters composed of>3 ESTs or≧1 RNA was found to be 4588.

Of the inter-contig regions identified having a length≧200 bp, 57% were classified as comprising unidirectional candidate DREs, putatively regulating expression of one flanking CDS (FIGS. 2a-b), and 22% were classified as comprising bidirectional candidate DREs, putatively regulating both flanking CDSs (FIG. 2c). The remaining 21% of the inter-contig regions were not classified as candidate DREs since these DREs were located downstream of both flanking contigs (“tail-to-tail DREs”). Thus, more than two-thirds of the candidate DREs identified were of the unidirectional type.

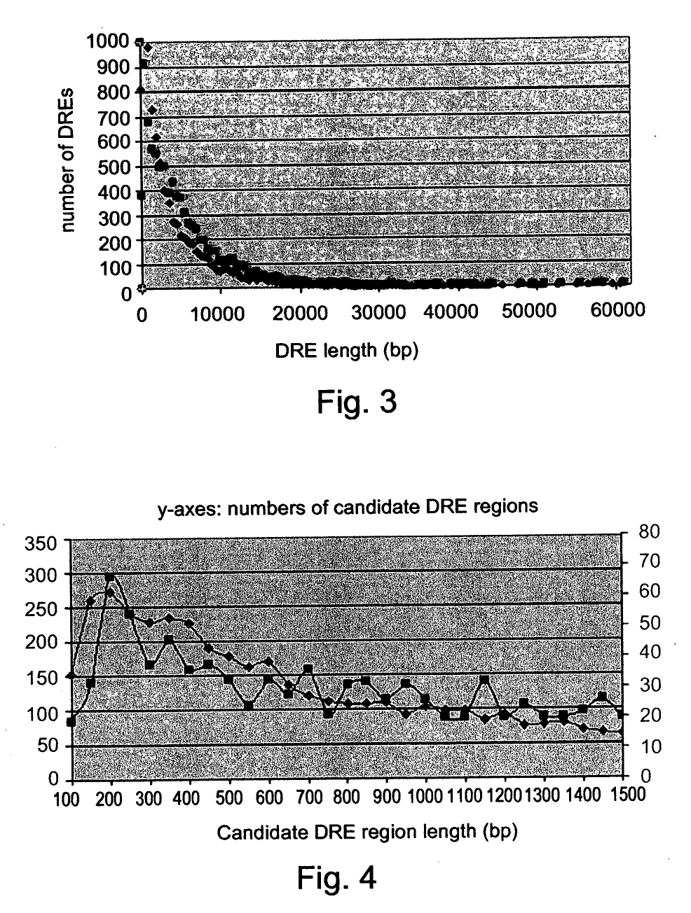

Numbers of unidirectional and bidirectional candidate DREs up to about 60,000 in length and 100-1,500 bp in length versus length of candidate DREs are shown in FIGS. 3 and 4, respectively. In both types of candidate DREs up to about 60,000 in length, the numbers of candidate DREs decline exponentially as their length increases (when viewing DRE lengths at the low candidate DRE length resolution of FIG. 3). Only 2% of the bidirectional candidate DREs (n=85) were found to be 100-200 bp in length.

Surprisingly, however, the numbers of candidate DREs having a length<200 bp drop sharply (FIG. 4). This may indicate that there is some biological or computational (LEADS) selection that may tend to minimize the length of candidate DREs.

These results can be confirmed only by check whether a set of DREs from this group will retain activity following molecular dissection thereof. This can be effected by checking whether contigs flanking bidirectional candidate DREs have similar expression patterns. It is hypothesized that bidirectional promoters will regulate similar expression patterns in both adjacent contigs.

These results demonstrate that the plant DRE sequence databases of the present invention constitute a novel, unique and potent tool which can be used to process information pertaining to plant DREs on a genomic scale and to greatly facilitate cloning of plant DREs. As such, the plant DRE sequence databases of the present invention can be exploited to great benefit in industries employing plants and plant products.

### Example 4

### Creation of an Annotated Database of Arabidopsis Candidate DRE Sequences Searchable according to Prioritizable Biological Parameters

The capacity to efficiently identify plant DREs having desired regulatory properties and the capacity to assign regulatory properties to a given non-coding nucleic acid sequence are highly desirable since such capacities can be exploited to great benefit in fields utilizing plants or plant products, for example by enabling the generation of genetically modified plants having desired characteristics. Thus, in order to provide such capacities, the present inventors have created an annotated database of Arabidopsis candidate DREs searchable according to a set of prioritizable biological parameters, as follows.

Materials and Methods:

Database Design:

The database of computationally identified plant candidate DRE sequences described above was annotated with the following data for each candidate DRE entry: DRE name, DRE origin, nucleotide sequence and orientation with respect to putatively regulated downstream sequences, biological parameter annotations corresponding to those of downstream CDSs (i.e., genes) and references to related patents. Biological annotations included corresponding gene name, gene products, functions of gene products, mutant phenotype, homologous genes, gene expression patterns, alternative splice variants and antisense RNA transcripts.

This annotated database was employed to analyze the genomic distribution of DREs with respect to various biological characteristics and to identify candidate DREs having selected biological characteristics.

Results:

Computational Identification of Arabidopsis Candidate DREs according to Selected Biological Parameters.

A total of 1,060 DREs were computationally selected and scored according to their probability of matching biological criteria including capacity to drive constitutive gene expression (i.e., strong expression in many parts of the plants), capacity to drive TF gene expression and capacity to drive organ specific expression.

Comparisons of 424 GenBank plant promoters to the computational internal-PCR products of all the DREs were performed yielded an alignment of 41 different known promoters to 104 different DREs. In some of these homologies the DREs were observed to contain more than 80% of the promoter, having an e-value of zero.

Thus, the databases of the present invention represent a novel and superior means with which to rapidly and efficiently identify novel DREs, such as plant DREs, having desired gene regulatory characteristics. Since the sequences of such DREs are provided by the databases of the present invention, these can be cloned and used to generate transgenic plants having desired characteristics which could be exploited to great benefit by industries plants or plant products having novel or desired characteristics.

### Example 5

### Rapid and Efficient Isolation of Plant DREs having Selected Biological Characteristics

As demonstrated above, the databases of the present invention can be utilized to provide the nucleic acid sequences of plant DREs having desired biological characteristics. The ability to efficiently isolate and clone such DREs is highly desirable since this enables construction of nucleic acid constructs capable of being used to generate transgenic plants possessing desired characteristics. Such a capacity is of enormous potential impact in industries employing plants or plant products. Thus, in order to enable the rapid and efficient isolation and cloning of the DREs of the present invention, the present inventors have computationally identified primer sequences suitable for the PCR amplification and cloning thereof and have annotated the DRE databases of the present invention with such primer sequences, as follows.

Materials and Methods:

PCR Amplification of Candidate DREs:

Candidate DREs listed in Table 7 were PCR amplified via nested PCR amplification of gDNA using two sets of primers computationally selected using PRIMER© software (EMBL) with modifications. A first, “external” pair of primers was designed to amplify a secondary template PCR product corresponding to a sequence extending 300-2000 bp upstream and downstream beyond the ends of candidate DRE sequences. A second, “internal”, pair of primers was designed to amplify from the secondary template PCR product a clonable PCR product comprising a sequence starting within 75 nucleotides upstream of the candidate DRE sequence and extending to within the first 100 nucleotides of the downstream-flanking transcribed contig sequence. Internal primers were each designed to contain a unique restriction site so as to enable cloning of the final PCR product in the proper orientation in an expression vector for driving reporter gene expression.

Experimental Results:

PCR Amplification of DREs Using Computationally Selected Primers:

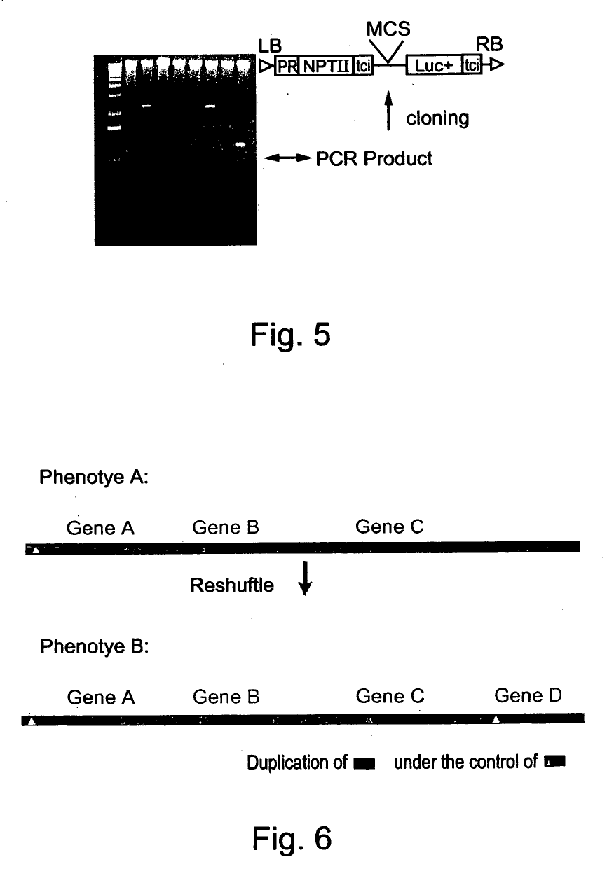

All primers designed and tested were found to efficiently PCR amplify sequences comprising target DREs. A representative electrophoretic analysis of an amplified DRE is shown in FIG. 5.

These results therefore demonstrate that the computationally selected primers provided by the databases of the present invention can be utilized to PCR amplify plant DREs having selected biological characteristics. Furthermore, the method of the present invention enables amplification of such candidate DRE sequences in such a way as to enable functional cloning thereof in expression vectors capable of being used to generate transgenic plants possessing desired characteristics.

TABLE 7
Examples of DREs computationally selected and
prioritized according to biological parameters.

| Type of gene | | | | | | | No. of ESTs |
|---|---|---|---|---|---|---|---|
| expression | Tissue specificity | | | | | | clustered in |
| putatively | of gene expression | | | Candidate | | | contig |
| driven by | putatively | Candidate | | DRE | | | located |
| candidate | regulated by | DRE | | length | No. of | Downstream | downstream |
| DRE | candidate DRE¹ | ID no.² | Score³ | (bp) | RNAs⁴ | contig ID no. | of DRE |
| Constitutive | above ground | 10179* | 2145 | 1078 | 0 | Z29720 | 195 |
| (maximum | organs, mainly | | | | | | |
| score: 1,724) | flowers | | | | | | |
| | above ground | 3714* | 1098 | 511 | 0 | Z25961 | 122 |
| | organs, mainly | 3560* | 1431 | 3147 | 1 | Z17937 | 158 |
| | root, siliques | | | | | | |
| | above ground | 22397* | 1310 | 1338 | 1 | ATHD12AAA | 131 |
| | organs, mainly | 1114* | 900 | 2831 | 0 | Z17940 | 100 |
| | root, seed, | | | | | | |
| | above ground | 24291* | 1278 | 2096 | 0 | Z17960 | 142 |
| | organs, mainly | 22252* | 840 | 3085 | 0 | Z17542 | 91 |
| | root, seed, siliques | | | | | | |
| Organ- | siliques | 2273 | 1125** | 3178 | 0 | AV556993 | 25 |
| specific | seed | 9634 | 700** | 1131 | 0 | BE521094 | 14 |
| (maximum | | 13640 | 250** | 3132 | 0 | BE521013 | 5 |
| score: 2,390) | flower | 20558 | 500** | 3167 | 1 | F15160 | 5 |
| | | 18818 | 300** | 3274 | 0 | AV531882 | 6 |
| | flower buds | 6039 | 390** | 3010 | 1 | Z25514 | 4 |
| | root | 14591 | 300** | 3140 | 0 | AI996687 | 5 |
| TF⁵ + organ | siliques | 24584 | 90** | 1952 | 1 | ATHAGL5A | 2 |
| specific | | | | | | | |
| TF⁵ | above ground | 1927 | | 242 | 1 | D58424 | 4 |
| | organs | | | | | | |

¹corresponds to the tissue source of the ESTs used to generate the contig whose expression is putatively regulated by the candidate DRE
²internal ID number assigned by the present inventors
³Computational score: a relative score enabling prioritization of contigs from most to least relevant with respect to specific parameters.
⁴Number of RNA molecules comprised in set of clustered sequences used to define contig putatively regulated by candidate DRE.
⁵There is no numeric score for TFs.
*constitutivity
**specificity

### Example 6

### Tissue- and Inductive Condition-Specific Expression of a Reporter Gene Under the Control of a Computationally Selected DRE in Arabidopsis Plants

The ability to genetically modify plants with DREs is highly desirable since these can be used to generate novel and/or selected gene expression patterns, thereby greatly facilitating the production of plants having novel and/or selected characteristics. For example, novel patterns of gene expression can be obtained by combinatorial shuffling of heterologous DRE-structural gene pairs within a genome (FIG. 6). In order to efficiently provide such a capacity with genomic scope, the present inventors have created DRE databases annotated with computationally selected primers capable enabling the identification and cloning of DREs having selected regulatory properties and have generate transgenic plants expressing transgenes with a selected pattern of gene expression therewith, as described hereinbelow.

Materials and Methods:

Cloning of Candidate DREs in Luciferase Reporter Gene Expression Vectors:

Candidate DREs computationally selected for driving high and constitutive, organ specific, or TF gene expression (listed in Table 7, above) were PCR amplified and cloned into a luciferase reporter gene expression vector, as depicted in FIG. 5, using the binary vector pBI101 (Clontech, USA).

Plant Growth and Transformation:

Arabidopsis plants were grown and transformed using the constructs described above via a high throughput dipping protocol, as previously described (Clough S J. and Bent A F. (1998) The Plant J. 16(6):735; Desfeux C. Plant Physiology 2000, 123:895), with minor modifications. Briefly, soil mixtures were mixed and irrigated immediately prior to sowing single plants in 250 ml pots. After sowing, pots covered with aluminum foil and plastic covers were incubated at 4° C. for 3-4 days prior to being transferred to a growth chamber at 18-24° C. with a 16 h/8 h on/off light cycle.

Transformations were performed using transformation medium at pH 5.7 containing 0.5 MS (2.15 g/l), 0.044 μM BAP, 112 μg B5 Gambourg vitamins, 5% sucrose, 200 μl/L Silwet L-77, 18.2 EC double-distilled water.

Luciferase Imaging:

Transformed Arabidopsis plantlets at a development stage of 2-3 true leaves were subjected to luminescence assays for detection of luciferase activity, as previously described (Meissner R., Plant J. 2000, 22:265) in a darkroom using an ultra-low light detection camera (Princeton Instruments Inc., USA).

Experimental Results:

Luciferase assays of transformed Arabidopsis plantlets identified DRE #10179 as driving high and constitutive gene expression in all parts of the plantlets, DRE #3714 as driving high and constitutive gene expression in flower buds, DRE #1927 as driving strong gene expression in all leaves, and DRE #24584 as driving strong TF gene expression mainly in the cotyledons. These results, demonstrated the capability of the bioinformatics procedures of the present invention to correctly identify DREs, to create a database from which these can be selected according to desired biological criteria and to design primers capable of amplifying these DREs. These results further demonstrated the capacity afforded by the methods described herein to rationally design and generate plants having novel and desired phenotypes resulting from modifications in gene regulation.

### Example 7

### “One-Tube” Method of Cloning DREs

A high throughput method of cloning DREs using a single reaction tube, referred to herein as the “one-tube” method, was developed in order to enable large scale production of DRE transformed transgenic plants.

Materials and Methods:

One-Tube Method of Cloning DREs in Binary Vectors:

DREs are cloned as follows. Arabidopsis thaliana (var col 1) leaf gDNA is extracted using DNAeasy Plant Mini Kit (Qiagen, Germany). Primers for PCR amplification of DREs are designed using PRIMER© software and modified to contain restriction sites. absent from the DRE sequence, for PCR product insertion into pVER1 binary plasmid.

Polymerase chain reaction analyses are performed using Taq Expand Long Template PCR kit (Roche), according to the manufacturer's instructions, using as thermal cycle: 92° C./2 min→10×[9420 C./10 min→55° C./30 sec→68° C./5 min]→18×[94° C./10 min→55° C./30 sec→22 68° C./5 min (+20 sec each cycle)]→68° C./7 min. PCR products are double-digested with restriction endonucleases according to the protocols described in Table 8.

TABLE 8
Candidate DRE double digestion protocols.

| | | | Digest | Heat | | | Digest | Heat |
|---|---|---|---|---|---|---|---|---|
| Enzyme | First | Buffer | time | inactivation | Second | | time | inactivation |
| combination | digest | (Roche) | (min) | conditions | digest | Buffer | (min) | conditions |
| HindIII, SalI | HindIII | M | 90 | 20 min, | SalI | M + NaCl + Tris | 60 | 20 min, |
| | | | | 70° C. | | | | 70° C. |
| HindIII, | HindIII | B | 30 | No | BamHI | B | 60 | 20 min, |
| BamHI | | | | | | | | 70° C. |
| SalI, BamHI | BamHI | M | 60 | 20 min, | SalI | M + NaCl + Tris | 60 | 20 min, |
| | | | | 80° C. | | | | 70° C. |
| HindIII, | HindIII | B | 30 | No | EcoRV | B | 60 | 20 min, |
| EcoRV | | | | | | | | 70° C. |
| SalI, ScaI | SalI, ScaI | H | 60 | 20 min, | | | | |
| | | | | 80° C. | | | | |
| BamHI, SmaI | SmaI | A | 60 | 20 min, | BamHI | A | 60 | 20 min, |
| | | | (30° C.) | 70° C. | | | | 80° C. |
| SalI, PvuII | PvuII | M | 60 | 20 min, | SalI | M + NaCl + Tris | 60 | 20 min |
| | | | | 80° C. | | | | |
| HindIII, | HindIII | M | 30 | No | PvuII | M | 60 | 20 min, |
| PvuII | | | | | | | | 80° C. |
| HindIII, StuI | HindIII, | B | 90 | 20 min, | | | | |
| | StuI | | | 80° C. | | | | |
| BamHI, StuI | StuI | B | 30 | No | BamHI | B | 60 | 20 min, |
| | | | | | | | | 80° C. |

Plasmid vector pVer1, derived from binary vector pBI101 (Clontech), is double-digested using the same restriction endonucleases used to excise cloned DREs from vector, purified using PCR Purification Kit (Qiagen, Germany), treated with alkaline-phophatase (Roche) according to the manufacturer's instructions and re-purified using PCR Purification Kit (Qiagen, Germany). Insertion of DRE into vector pVer1 vector is performed by adding to DRE digests: 500 ng of double digested pVer1 plasmid, 1 μl of T4 DNA ligase (40 U/μl; Roche) and 6 μl of T4 buffer (Roche). Following overnight incubation of ligation mixes at 4° C., Agrobacterium tumefaciens GV303 competent cells are transformed using 1-2 μl of ligation reaction mixture by electroporation, using a MicroPulser electroporator (Biorad), 0.2 cm cuvettes (Biorad) and EC-2 electroporation program (Biorad). Agrobacterium cells are grown on LB at 28° C. for 3 h and plated on LB-agar plates supplemented with the antibiotics gentamycin 50 mg/L (Sigma) and kanamycin 50 mg/L (Sigma). Plates are then incubated at 28° C. for 48 h. Cloned DREs are identified by PCR analysis of bacterial colony DNA using the vector specific, insert flanking upstream and downstream primers 5′-AGGTACTTGGAGCGGCCGCA-3′ (SEQ ID NO:1) and 5′-CGAACACCACGGTAGGCTG-3′ (SEQ ID NO:2), respectively and the thermal cycle: 92° C./3 min→31×[94° C./30 sec→54° C./30 sec→72° C./X min (X=length (kb) of longest PCR product expected)]→72° C./10 min. Positive Agrobacterium colonies are subsequently used for Arabidopsis plant transformation.

### Example 8

### Validation of Computational Identification of Contig, CDS and Candidate DRE Sequences and Annotation of Databases Thereof

As described above, the present invention enables computational identification of candidate DREs and assignment of regulatory capacities thereto. In order to validate such computational candidate DRE identification and assignment of function, the present inventors utilize a broad range of verification methods, as follows.

Materials and Methods:

Promine Analysis:

Computationally identified candidate DREs selected for biological validation are PCR amplified using computationally selected primers, cloned into binary vectors having luciferase reporter genes, and the resultant vectors are transformed into Arabidopsis plants (5 plants are transformed with each construct. Seeds from transformed plants are harvested and sown on plates using growth medium containing kanamycin as a selection marker. Antibiotic resistant transformants are grown and 10 T1 plants are kept per construct. T2 seeds from each plant are collected and grown in the presence of kanamycin and mature plants are analysed with luciferin.

Manual Data Annotation:

Accurate and exhaustive manual annotation of data is used to optimize annotation of expressed sequence databases and candidate DRE sequence databases. For example, accurate classification of tissue, developmental stage and/or growth condition specificity of libraries from which clustered ESTs are derived ensures accurate annotation of expressed contig and candidate DRE sequences with respect to such biological characteristics.

Biological Specificity of Contigs, CDSs and Candidate DREs:

The percentage of clustered ESTs which define a contig or a CDS, and are uniquely specific to a given type of EST library is correlated with the probability that such contigs or CDSs are specifically expressed in cells whose tissue-, developmental stage-, and/or growth condition-specificity correspond to those of the cells from which such a library is derived. Conversely, the percentage of clustered ESTs which define a contig or a CDS, and are specific to multiple types of EST libraries is correlated with the probability that such contigs or CDSs are constitutively expressed.

Clustering Quality:

The number of ESTs assigned high interest scores, and used to define contigs, CDSs or candidate DREs is correlated with the probability that the nucleotide sequences of such contigs, CDSs or candidate DREs are accurate and that these contigs, CDSs or candidate DREs are indeed specific to the tissue of interest.

Inducibility:

Confirmation that contigs, CDSs or candidate DREs are comprised in inducible genes is obtained via text mining.

Candidate DRE Quality Assurance:

To assist in confirming that inter-contig regions indeed comprise candidate DREs, DRY analysis was performed via TBLASTX homology analysis of candidate DRE internal PCR product sequences (see Example 5, above).

Internal PCR products rather than whole candidate DREs were analyzed since the former are cloned in vectors and, as such, this enables quality assurance at a level further downstream in the processing of DREs than that of the LEADS algorithm.

Verification that inter-contig regions correspond as expected to non-transcribed regions of the genome was performed by verifying the absence of expressed homologs thereof in GenBank nr, with significant homology being defined by a cut-off e-score of<10⁻⁴.

Homology of Candidate DREs to Known Promoters:

External database homology searches were performed in order to identify candidate DREs homologous to known promoter sequences.

Results:

Candidate DRE Quality Assurance:

Searches of GenBank nr identified 3836 transcribed nucleic acid sequences comprising regions being homologous to those of inter-contig regions which could be sorted into 3 categories (“True”, “Mixed” and “False”), as follows.

“True” DREs:

Inter-contig regions comprise non-transcribed nucleic acid sequences as well as, at their upstream ends, portions of expressed sequences actually belonging to the flanking contig. This can result from such expressed sequences not being listed in external databases as a result of incomplete or unsuccessful sequencing. Hence, such candidate DREs (comprising regions of≧200 bp in length not homologous to transcribed nucleic acid sequences) retained their candidate DRE status and are used without modification to regulate heterologous gene expression.

“Mixed” DREs:

Inter-contig regions comprise, at their downstream ends, sequences found to be homologous to transcribed nucleic acid sequences listed in external databases. Such inter-contig regions retained their status as comprising candidate DREs but transcribed portions thereof are removed to regulate expression of heterologous genes fused downstream.

“False” DREs.

Homologies to transcribed nucleic acid sequences are due to both contigs flanking the inter-contig sequence being in fact portions of a single gene, the inter-contig region therefore being a portion of this gene in the center thereof. Such inter-contig regions are artifacts resulting from sets of clustered ESTs discontinuously representing an expressed sequence and were therefore de-classified as being candidate DREs. In any case of uncertainty, inter-contig regions were classified as “False”.

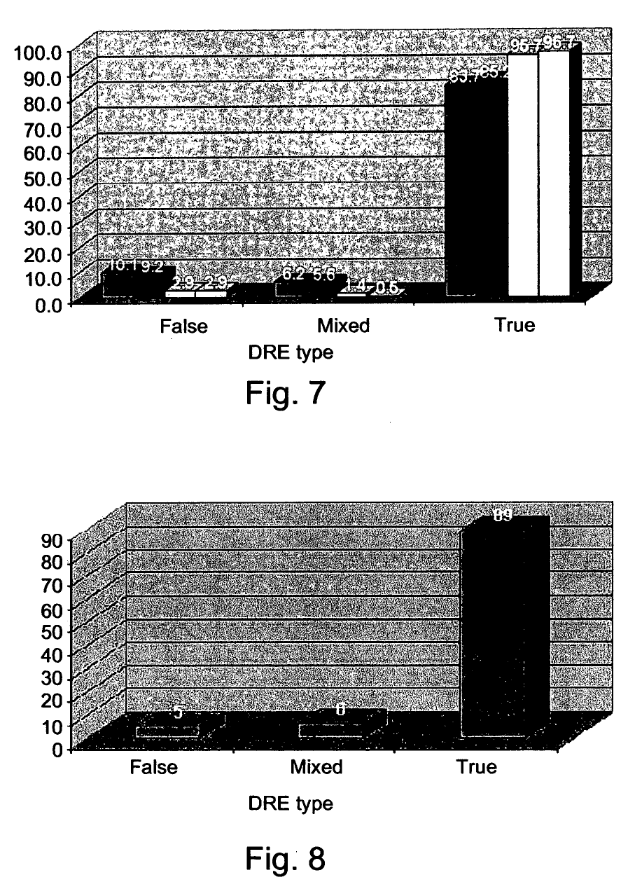

The percentages of “True”, “False” and “Mixed” DREs out of all inter-contig regions identified (16,176), and out of the inter-contig regions remaining (4,588) after discarding inter-contig regions upstream of contigs assembled from clusters comprising<4 ESTs or zero mRNAs are shown in FIG. 7. These results show that in both cases≧90% of the inter-contig regions comprised candidate DREs, ≧84% of which being classified as “True”. Discarding of such clusters was found to improve results by about 1%.

Homology of Contig Sequences Flanking Candidate DREs with Known RNA Sequences:

Homology of one (210 candidate DREs) or both (69 candidate DREs) flanking contig sequences with known RNA sequences was found to increase the percentages of inter-contig regions confirmed as comprising candidate DREs to 96 and 97%, respectively, out of all inter-contig regions identified, and out of the inter-contig regions remaining after discarding inter-contig regions upstream of contigs assembled from clusters comprising<4 ESTs or no mRNAs (FIG. 7).

As shown in FIG. 7, homology of one or both candidate DRE flanking contig sequences with known RNA sequences was found to increase the percentage of candidate DREs classified as “True” by 7%.

Quality of DREs Selected According to Biological Parameters:

As shown in FIG. 8, the large majority (89%) of 150 DREs selected for functional analysis were classified as “True” candidate DREs. Only 9 (5%) were classified as “False” candidate DREs. These were used as negative controls.

DREs were selected according to the quality of downstream contigs, as described above; strong expression, constitutive pattern of expression, organ specificity, interest score of downstream contigs, bidirectionality and TF function.

Homology of Candidate DREs to Known Promoters:

Database homology searches identified candidate DREs having homology to known promoter sequences.

Following e-score filtering, database searches identified 55 candidate DREs having homology to known promoter sequences, such candidate DREs putatively regulating: 5 TFs (identified via Pfam), 7 tissue-specific contigs, 4 constitutively expressed contigs and 4 quality contigs (satisfying clustering criteria, as described above and having high subjective interest scores).

Candidate DREs identified as being homologous to known promoters included candidate DREs putatively regulating contig ATU13949, 100% homologous (e-score=0) to a known heat-shock protein; contig Z34226, homologous (e-score=6×10⁻¹³) to a low-temperature-induced protein; and contig Z34203, homologous (e-score=1×10⁻¹⁴) to an S-adenosyl-methionine-sterol-C-methyltransferase.

The quality of the computational DRE databases of the present invention is verified via positive control homology searches against biologically validated Arabidopsis promoters to see whether these promoters exist in the DRE database of the present invention. Positive control homology searches of the computational DRE databases of the present invention are performed against all known sequences annotated as plant promoters in GenBank. Alternatively, homology searches of the computational DRE databases of the present invention against GenBank nr are performed so as to detect inter-contig regions which are actually coding regions.

External database searches identified known promoter sequences homologous to those of candidate DRE sequences corresponding to those of internal PCR products (see Example 5, above).

Statistical Analysis:

Out of 424 sequences of known plant promoters which were compared using TBLASTX to those of the internal PCR product sequences (see Example 5, above) of all candidate DREs, 41 were found to be homologous to 104 different candidate DREs. All but 7 of the homologous promoter sequences were homologous to≦3 candidate DREs therefore these promoter sequences are unlikely to be repeats or transposons.

These results therefore demonstrate that the present invention provides for varied and highly efficient methods of verifying correct identification and processing of expressed and regulatory sequences of plants at the genome level. As such, the databases of the present invention provide a reliable and powerful tool, far superior to all prior art methods, for producing plants having desired characteristics.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. A method of generating genotypic and possibly phenotypic variation in an organism comprising:
(a) isolating at least one non-coding nucleic acid sequence from a genome of the organism; and
(b) genetically transforming the organism with said at least one non-coding nucleic acid sequence to thereby generate genotypic and possibly phenotypic variation in the organism.

2. The method of claim 1, wherein said at least one non-coding nucleic acid sequence is isolated from an inter-contig region of said genome.

3. The method of claim 1, wherein said organism is a plant.

4. The method of claim 1, wherein isolating said at least one non-coding nucleic acid sequence is effected by:
(i) computationally clustering transcribed nucleic acid sequences of the organism to thereby obtain a plurality of clusters;
(ii) computationally generating contigs from at least a subset of said plurality of clusters;
(iii) computationally aligning said contigs with the genomic nucleic acid sequences of the organism to thereby identify inter-contig region sequences of the genome of the organism; and
(iv) amplifying at least one of said inter-contig region sequences to thereby obtain said at least one isolated non-coding nucleic acid sequence.

5. The method of claim 4, wherein said transcribed sequences are selected from the group consisting of EST sequences, cDNA sequences, mRNA sequences and preanalyzed genomic sequences.

6. The method of claim 4, further comprising assigning to said contigs a score according to at least one parameter selected from the group consisting of:
(a) the number of said transcribed nucleic acid sequences clustered;
(b) the percent homology of nucleotide sequences of said contigs to nucleotide sequences of known transcription factors;
(c) the percent homology of nucleotide sequences of said contigs to nucleotide sequences of selected genes of interest;
(d) the number of expression libraries from which said contigs were generated;
(e) the number of types of expression libraries from which said contigs were generated;
(f) the number of RNAs comprised in said plurality of clusters;
(g) the length of the contig;
(h) a user-defined quality score;
(i) the type of tissues from which said transcribed nucleic acid sequences were derived;
(j) the developmental stage of the tissues from which said transcribed nucleic acid sequences were derived
(k) the growth conditions of the tissue from which said transcribed nucleic acid sequences were derived; and
(l) the number of clusters of said transcribed nucleic acid sequences generated by the library from which said contigs are derived.

7. A method of identifying novel gene expression regulatory sequences comprising:
(a) isolating at least one non-coding nucleic acid sequence from a genome of an organism;
(b) transforming said organism with an expression cassette including said at least one non-coding nucleic acid sequence covalently linked to a reporter nucleic acid sequence; and
(c) monitoring reporter activity, said reporter activity being indicative of a presence of a regulatory sequence in said at least one non-coding nucleic acid sequence.

8. The method of claim 7, wherein said expression cassette further includes a promoter sequence upstream of said reporter nucleic acid sequence.

9. The method of claim 7, wherein said organism is a plant.

10. The method of claim 7, wherein isolating said at least one non-coding nucleic acid sequence is effected by:
(i) computationally clustering transcribed nucleic acid sequences of the organism to thereby obtain a plurality of clusters;
(ii) computationally generating contigs from at least a subset of said plurality of clusters;
(iii) computationally aligning said contigs with the genomic nucleic acid sequences of the organism to thereby identify inter-contig region sequences of the genome of the organism; and
(iv) amplifying at least one of said inter-contig region sequences to thereby obtain said at least one isolated non-coding nucleic acid sequence.

11. The method of claim 10, wherein said transcribed nucleic acid sequences are selected from the group consisting of EST sequences, cDNA sequences, mRNA sequences and preanalyzed genomic sequences.

12. The method of claim 10, further comprising assigning to said contigs a score according to at least one parameter selected from the group consisting of:
(a) the number of said transcribed nucleic acid sequences clustered;
(b) the percent homology of nucleotide sequences of said contigs to nucleotide sequences of known transcription factors;
(c) the percent homology of nucleotide sequences of said contigs to nucleotide sequences of selected genes;
(d) the number of expression libraries from which said contigs were generated;
(e) the number of types of expression libraries from which said contigs were generated;
(f) the number of RNAs comprised in said plurality of clusters;
(g) the length of the contig;
(h) the types of methods whereby said transcribed nucleic acid sequences were derived;
(i) the type of tissues from which said transcribed nucleic acid sequences were derived;
(j) the developmental stage of the tissues from which said transcribed nucleic acid sequences were derived
(k) the growth conditions of the tissue from which said transcribed nucleic acid sequences were derived; and
(l) the number of clusters of said transcribed nucleic acid sequences generated by the library from which said contigs are derived.

13. A method of generating a database of putative regulatory sequences of a genome of an organism comprising:
(a) computationally clustering transcribed nucleic acid sequences of the organism to thereby obtain a plurality of clusters;
(b) computationally generating contigs from at least a subset of said plurality of clusters;
(c) computationally aligning said contigs with the genomic nucleic acid sequences of the organism to thereby obtain inter-contig region sequences of the genome of the organism; and
(d) storing said inter-contig region sequences of the genome of the organism in a database.

14. The method of claim 13, further comprising:
(e) computationally clustering said inter-contig region sequences of the genome of the organism to thereby identify and group non-redundant sequences.

15. The method of claim 13, further comprising assigning to said contigs a score according to at least one parameter selected from the group consisting of:
(a) the number of said transcribed nucleic acid sequences clustered;
(b) the percent homology of nucleotide sequences of said contigs to nucleotide sequences of known transcription factors;
(c) the percent homology of nucleotide sequences of said contigs to nucleotide sequences of selected genes;
(d) the number of expression libraries from which said contigs were generated;
(e) the number of types of expression libraries from which said contigs were generated;
(f) the number of RNAs comprised in said plurality of clusters;
(g) the length of the contig;
(h) the types of methods whereby said transcribed nucleic acid sequences were derived;
(i) the type of tissues from which said transcribed nucleic acid sequences were derived;
(j) the developmental stage of the tissues from which said transcribed nucleic acid sequences were derived
(k) the growth conditions of the tissue from which said transcribed nucleic acid sequences were derived; and
(l) the number of clusters of said transcribed nucleic acid sequences generated by the library from which said contigs are derived.

16. A computer readable media comprising as retrievable records data pertaining to a plurality of nucleic acid sequences, each of said plurality of nucleic acid sequences representing an inter-contig region sequence of a genome of a single organism.

17. A nucleic acid construct library comprising a plurality of nucleic acid constructs each including a specific non-coding nucleic acid sequence of an organism and devoid of coding sequences of said organism.

18. The nucleic acid construct library of claim 17, wherein each of said plurality of said nucleic acid constructs further includes a coding nucleic acid sequence of a known protein covalently linked to said specific non-coding nucleic acid sequence.

19. A method of determining the minimal number of expressed sequence tags (ESTs) needed for constructing substantially all of the coding sequences of a genome of an organism, the method comprising:
(a) predicting the number of genes present in the genome of the organism, said number of genes being represented by N;
(b) obtaining a product of N(ln(N)+C), wherein C=0.5772, said product being the minimal number of ESTs needed for constructing substantially all of the coding sequences of a genome of an organism.

20. A kit comprising a plurality of primer pairs, each of said primer pairs being complementary with nucleic acid sequences flanking a specific inter-contig region sequence of a genome of an organism, such that the kit being useful for amplifying a plurality of inter-contig region sequences of said genome of said organism.

21. A method of identifying putative regulatory sequences comprising:
(a) computationally identifying inter-contig region sequences of at least two distinct organisms; and
(b) computationally comparing said inter-contig region sequences of said at least two distinct organisms to thereby identify non-redundant sequences, said non-redundant sequences being putative regulatory sequences.

22. The method of claim 21, wherein said at least two distinct organisms represent closely related species.

23. A computing platform for identifying inter-contig region. sequences of an organism and for generating primer sequences for amplifying said inter-contig region sequences, the computing platform comprising a processing unit being for:
(a) computationally comparing data pertaining to transcribed nucleic acid sequences of an organism with data pertaining to genomic sequences of the organism to thereby generate data pertaining to inter-contig sequences of the organism; and
(b) automatically generating primer sequences suitable for amplifying said inter-contig sequences of the organism.

24. A method of generating genotypic and possibly phenotypic variation in an organism comprising:
(a) isolating at least one non-coding nucleic acid sequence from a genome of the organism;
(b) covalently linking said at least one non-coding nucleic acid sequence to a known coding sequence to thereby generate an expression cassette; and
(b) genetically transforming the organism with said expression cassette to thereby generate genotypic and possibly phenotypic variation in the organism.

25. The method of claim 24, wherein the organism is a plant.

26. The method of claim 24, wherein isolating said at least one non-coding nucleic acid sequence is effected by:
(i) computationally clustering transcribed nucleic acid sequences of the organism to thereby obtain a plurality of clusters;
(ii) computationally generating contigs from at least a subset of said plurality of clusters;
(iii) computationally aligning said contigs with the genomic nucleic acid sequences of the organism to thereby identify inter-contig region sequences of the genome of the organism; and
(iv) amplifying at least one of said inter-contig region sequences to thereby obtain said at least one isolated non-coding nucleic acid sequence.

27. The method of claim 24, wherein said transcribed nucleic acid sequences are selected from the group consisting of EST sequences, cDNA sequences, mRNA sequences and preanalyzed genomic sequences.

28. The method of claim 26, further comprising assigning to said contigs a score according to at least one parameter selected from the group consisting of:
(a) the number of said transcribed nucleic acid sequences clustered;
(b) the percent homology of nucleotide sequences of said contigs to nucleotide sequences of known transcription factors;
(c) the percent homology of nucleotide sequences of said contigs to nucleotide sequences of selected genes;
(d) the number of expression libraries from which said contigs were generated;
(e) the number of types of expression libraries from which said contigs were generated;
(f) the number of RNAs comprised in said plurality of clusters;
(g) the length of the contig;
(h) the types of methods whereby said transcribed nucleic acid sequences were derived;
(i) the type of tissues from which said transcribed nucleic acid sequences were derived;
(j) the developmental stage of the tissues from which said transcribed nucleic acid sequences were derived
(k) the growth conditions of the tissue from which said transcribed nucleic acid sequences were derived; and
(l) the number of clusters of said transcribed nucleic acid sequences generated by the library from which said contigs are derived.

29. A method of uncovering regulatory sequences functional in a biological pathway of an organism, the method comprising:
(a) isolating non-coding nucleic acid sequences from a genome of the organism;
(b) covalently linking each of said non-coding nucleic acid sequences to a reporter coding sequence to thereby generate a plurality of expression cassettes;
(c) genetically transforming a plurality of organisms with said plurality of said expression cassettes;
(d) inducing activation of the biological pathway in said plurality of organisms; and
(e) monitoring reporter activity in said plurality of organisms prior to, and following, step (d), to thereby determine the presence or absents of a regulatory sequence functional in the biological pathway in each of said non-coding nucleic acid sequences.

30. The method of claim 29, wherein the organism is a plant.

31. The method of claim 29, wherein isolating said non-coding nucleic acid sequences is effected by:
(i) computationally clustering transcribed nucleic acid sequences of the organism to thereby obtain a plurality of clusters;
(ii) computationally generating contigs from at least a subset of said plurality of clusters;
(iii) computationally aligning said contigs with the genomic nucleic acid sequences of the organism to thereby identify inter-contig region sequences of the genome of the organism; and
(iv) amplifying said inter-contig region sequences to thereby obtain said isolated non-coding nucleic acid sequences.

32. The method of claim 31, wherein said transcribed nucleic acid sequences are selected from the group consisting of EST sequences, cDNA sequences, mRNA sequences and preanalyzed genomic sequences.

33. The method of claim 31, further comprising assigning to said contigs a score according to at least one parameter selected from the group consisting of:
(a) the number of said transcribed nucleic acid sequences clustered;
(b) the percent homology of nucleotide sequences of said contigs to nucleotide sequences of known transcription factors;
(c) the percent homology of nucleotide sequences of said contigs to nucleotide sequences of selected genes;
(d) the number of expression libraries from which said contigs were generated;
(e) the number of types of expression libraries from which said contigs were generated;
(f) the number of RNAs comprised in said plurality of clusters;
(g) the length of the contig;
(h) the types of methods whereby said transcribed nucleic acid sequences were derived;
(i) the type of tissues from which said transcribed nucleic acid sequences were derived;
(j) the developmental stage of the tissues from which said transcribed nucleic acid sequences were derived
(k) the growth conditions of the tissue from which said transcribed nucleic acid sequences were derived; and
(l) the number of clusters of said transcribed nucleic acid sequences generated by the library from which said contigs are derived.

34. A method of generating phenotypic variation in an organism comprising:
(a) isolating non-coding nucleic acid sequences from a genome of the organism;
(b) generating a plurality of organisms genetically transformed with said non-coding nucleic acid sequences; and
(c) isolating an organism of said plurality of organisms which exhibits phenotypic variation.

35. The method of claim 34, further comprising the step of culturing said plurality of organisms genetically transformed with said non-coding nucleic acid sequences under conditions suitable for identifying said phenotypic variation.

36. The method of claim 34, wherein the organism is a plant.

37. The method of claim 34, wherein isolating said non-coding nucleic acid sequences is effected by:
(i) computationally clustering transcribed nucleic acid sequences of the organism to thereby obtain a plurality of clusters;
(ii) computationally generating contigs from at least a subset of said plurality of clusters;
(iii) computationally aligning said contigs with the genomic nucleic acid sequences of the organism to thereby identify inter-contig region sequences of the genome of the organism; and
(iv) amplifying said inter-contig region sequences to thereby obtain said isolated non-coding nucleic acid sequences.

38. The method of claim 37, wherein said transcribed nucleic acid sequences are selected from the group consisting of EST sequences, cDNA sequences, mRNA sequences and preanalyzed genomic sequences.

39. The method of claim 37, further comprising assigning to said contigs a score according to at least one parameter selected from the group consisting of:
(a) the number of said transcribed nucleic acid sequences clustered;
(b) the percent homology of nucleotide sequences of said contigs to nucleotide sequences of known transcription factors;
(c) the percent homology of nucleotide sequences of said contigs to nucleotide sequences of selected genes;
(d) the number of expression libraries from which said contigs were generated;
(e) the number of types of expression libraries from which said contigs were generated;
(f) the number of RNAs comprised in said plurality of clusters;
(g) the length of the contig;
(h) the types of methods whereby said transcribed nucleic acid sequences were derived;
(i) the type of tissues from which said transcribed nucleic acid sequences were derived;
(j) the developmental stage of the tissues from which said transcribed nucleic acid sequences were derived
(k) the growth conditions of the tissue from which said transcribed nucleic acid sequences were derived; and
(l) the number of clusters of said transcribed nucleic acid sequences generated by the library from which said contigs are derived.

40. The method of claim 34, further comprising covalently linking a coding sequence of a known protein to each of said non-coding nucleic acid sequences prior to step (b).

41. A method of generating phenotypic variation in an organism comprising:
(a) isolating non-coding nucleic acid sequences from a genome of the organism;
(b) combinatorially shuffling regions derived from said non-coding nucleic acid sequences, to thereby generate combinatorial non-coding nucleic acid sequences;
(b) generating a plurality of organisms genetically transformed with said combinatorial non-coding nucleic acid sequences; and
(c) isolating an organism of said plurality of organisms which exhibits phenotypic variation.

42. The method of claim 41, further comprising generating a plurality of organisms genetically transformed with said non-coding nucleic acid sequences, isolating a non-coding nucleic acid sequence from each organism which exhibits phenotypic variation and using isolated non-coding nucleic acid sequences for said combinatorial shuffling of step (b).

43. The method of claim 41, further comprising characterizing said non-coding nucleic acid sequences prior to step (b).


SEQUENCE LISTING (2 sequences)

SEQ ID NO: 1 - DNA, 20 bp, Artificial sequence
  Synthetic single strand oligonucleotide
aggtacttggagcggccgca

SEQ ID NO: 2 - DNA, 19 bp, Artificial sequence
  Synthetic single strand oligonucleotide
cgaacaccacggtaggctg